(19) United States
(12) Patent Application Publication
(10) Pub. No.: US 2024/0112766 A1
(43) Pub. Date: Apr. 4, 2024

(54) SYSTEMS AND METHODS FOR REGISTERING AND REPORTING IN A TESTING PROGRAM

(71) Applicant: FloodLAMP Biotechnologies, PBC, Portola Valley, CA (US)

(72) Inventors: Randall James TRUE, Portola Valley, CA (US); Theresa Chia-Wen LING, Portola Valley, CA (US)

(73) Assignee: FloodLAMP Biotechnologies, PBC, Portola Valley, CA (US)

(21) Appl. No.: 18/338,828

(22) Filed: Jun. 21, 2023

Related U.S. Application Data
(63) Continuation-in-part of application No. 17/654,806, filed on Mar. 14, 2022.
Application No. 17/654,806 is a continuation of application No. PCT/US21/73022, filed on Dec. 19, 2021.
(60) Provisional application No. 63/199,369, filed on Dec. 21, 2020.
(60) Provisional application No. 63/354,660, filed on Jun. 22, 2022.
(60) Provisional application No. 63/366,836, filed on Jun. 22, 2022.

(51) Int. Cl. G16H 10/20; G16H 15/00

(52) U.S. Cl. CPC G16H 10/20; G16H 15/00

(57) ABSTRACT

Systems and methods for the recruitment, registration, or tracking of results associated with individuals who are in close contact with other individuals, for example, individuals who are organization/entity members, are provided. These systems and methods may utilize a mobile application and a cloud-based computing architecture to permit a user to register and identify relationships between individuals or identify relationships between individuals and an organization/entity. The systems and methods may enable generation of information related to registration status, testing participation information, test results, or information related to actions, decisions, or status related to a testing program. The systems and methods may enable an organization/entity to manage staffing or utilization of services based on data from a testing program.

### CROSS-REFERENCE TO RELATED APPLICATION

This application is a continuation-in-part of U.S. application Ser. No. 17/654,806, filed on Mar. 14, 2022 and published as U.S. Patent App. Pub. No. US20220208394A1 on Jun. 30, 2022, which is a continuation of International App. No. PCT/US2021/073022, filed on Dec. 19, 2021, which claims the benefit of priority to U.S. Provisional Patent Application No. 63/199,369, filed on Dec. 21, 2020; this application claims benefit of priority to U.S. Provisional Patent App. No. 63/366,836, filed on Jun. 22, 2022, and U.S. Provisional Patent App. No. 63/354,660, filed on Jun. 22, 2022. Each of the above-listed priority documents are hereby incorporated by reference herein in their entirety for all purposes.

### BACKGROUND

### Field of the Disclosure

This disclosure relates to the field of biological or environmental sample collection processes and sample testing and test reporting workflows.

The COVID-19 crisis has revealed many limitations of biospecimen testing in the United States and around the world. One key problem has been the lack of an adequate supply chain of critical reagents and supplies needed by testing labs to process samples. Other problems that have contributed to long turnaround times and high costs have been low throughput in sample collection, transport, and intake steps. “Samples are first collected, suspended in transport media, and then shipped in bulk to a testing facility. Bags of tens to hundreds of samples are delivered multiple times a day and lab workers must manually sort through them and enter relevant patient information into the facility's system.” [LGC Lessons on Sample Accessioning and Processing at Clinical Labs During a Pandemic (see: https://www.biosearchtech.com/)] The intake step, including the manual process of creating a digital record describing the collection of individual samples included in a pooled sample prior to testing the pooled sample, is a major bottleneck for pooled testing. This bottleneck is particularly significant as pooled testing offers the potential for significant efficiency gains in reduced usage of reagents and supplies, thus achieving larger coverage or increased frequency in disease testing/screening programs.

The COVID-19 pandemic has also revealed the need for significant progress in many aspects of public health and emergency response. Advances in biotechnology, such as the rapid development of mRNA vaccines have been highly successful. Another critical area, diagnostics and testing, has fallen far short of its potential. Testing provides essential information, both at the individual and at the societal levels. Current options in the testing space continue to be inadequate for containing the spread of a pandemic level pathogen such as SARS-CoV-2.

The most widely available testing options are either over-the-counter antigen tests or central lab-based PCR tests. Antigen tests provide convenience but greatly sacrifice accuracy. One of the best studies in the subject measured a 23% positive detection rate of a top antigen test compared to a standard PCR test (see: [https://doi.org/10.1101/2022.02.27.22271090]). PCR testing, though more accurate, requires an expensive instrument, and thus incentivizes the centralization of testing. Access to PCR testing has been hampered by long wait times for results and high costs. The central lab processing model has proven to be especially inadequate during times of infection surges, when testing is needed most.

New rapid at-home molecular tests marry speed, accuracy, and convenience, but are cost prohibitive for most people and circumstances. Fast, accessible, and accurate testing results are critical for managing spread, preventing outbreaks, and helping individuals and organizations to make appropriate decisions in the interest of public health. Newer isothermal amplification chemistries, e.g., Loop-mediated Isothermal Amplification (LAMP), have matured during the pandemic and offer great hope for the future. Because isothermal tests may run at a single temperature and can be configured to give a visual readout, instrumentation may not be needed, making a decentralized model possible.

New testing paradigms and efficiencies are needed in order to more holistically utilize testing in a way that helps to suppress transmission rather than to simply diagnose it. This is paramount for an aerosolized, asymptomatically transmitting virus. Progress in testing, including in many aspects of testing programs, would offer far reaching improvements to human health.

Testing programs are often implemented by organizations or entities, such as schools, businesses, or communities, and can offer on-demand testing, scheduled routine screening, or a combination of both. “Screening” usually refers to a type of testing for people who have no symptoms and no known, suspected, or reported close contact exposure, and helps to identify unknown infections that may cause additional infections. Screening may refer to repeated testing, and participants in a screening program may develop symptoms or have an exposure and be tested.

Frequently a goal of testing programs is to reduce spread within the organization and its interacting population. The two primary levers for reducing spread are the frequency of testing and sensitivity (accuracy) of the test. A more sensitive test offers more advance notice of when a person may be likely to infect others around them.

Very few testing programs routinely screen households due to the high cost and logistical demands. Intra-household transmission however is one of the main vectors for disease spread. In epidemiologic context, people within a household, such as a family, have a high degree of exposure to each other and are typically likely to transmit an infectious disease to each other. Further, each member of a household also has exposure to others they interact with in congregate settings outside the home, such as schools and workplaces. Testing the household together, either each person individually or with samples combined in a sample “pool,” provides an extra degree of assurance for an organization-based testing program.

The implementation of a testing program by an organization/entity often involves substantial overhead for administration and coordination of many aspects of the program, outside of the actual processing of test samples. This overhead may be a significant or even majority portion of the labor allocation or direct costs. Software usually plays a key role in testing programs and is often a substantial expense. Reducing the overhead through software improvements and administrative tools saves time and reduces the number of touchpoints, helping to reduce the overall burden for the organization/entity. Additionally, the user experience for participants in the testing program is a key consideration to encourage high participation rates in the testing program. Improved user experience leads to increased adoption, satisfaction, and compliance with program protocols—all improving the health and economic outcomes for both individuals and the organization/entity.

### SUMMARY OF THE DISCLOSURE

Biological sample collection and accessioning (intake) are some of the biggest bottlenecks in testing (e.g., human biological sample testing). Some of the disclosed embodiments address these bottlenecks through self-collection of pooled samples (e.g., via a mobile (e.g., smartphone) app). In some embodiments, participants register, typically through an organization such as their school, employer, or other community organization, and then collect samples together. In some embodiments, participants put 2 to 24 nasal swabs in one tube, with the sample collection process being tracked through a mobile app. In some embodiments, bagged pre-pooled samples are dropped off at collection points and picked up by program staff. In some embodiments, since the pooling happens outside the lab and in a distributed manner, large time and cost savings are reaped. In some embodiments, in combination with the sensitive test chemistry, this process permits frequent screening. In some embodiments, small pools of samples (e.g., 10 samples from a sample collection event) may be further pooled in the lab to 30, 50, or even 100 samples to be tested at once. This may be possible even with local infection rates above a few % because, in some embodiments, all program participants have previously tested negative. Getting larger and larger numbers of people in such a screening program protects the group from unknown community spread and drives down the disease prevalence. In some embodiments, the disclosed app may be widely used, including for use in screening schools to protecting students, teachers, and parents as in-person classes resume.

The disclosed embodiments include systems and methods for streamlined collection of samples that are, in some embodiments, combined together for pooled testing. In some embodiments, the disclosed systems and methods improve the efficiency of both sample collection programs and testing labs, leading to lower per sample costs and higher overall throughput of samples able to be tested. The majority of current “typical” testing involves a healthcare worker obtaining an individual specimen, such as a nasal swab or saliva, from the person being tested. This sample is put into a biohazard bag and sent to a lab for processing.

In some embodiments, the disclosed systems and methods describe testing programs that facilitate the decentralized collection of a large number of pooled samples through an app or a browser. In some embodiments, the disclosed testing program eliminates the need for the healthcare worker, instead utilizing self-collection which has been authorized by the FDA for a number of different sample kits. Further, in some embodiments, it enables participants in the testing program to pool their samples together and also creates the means to track that process so that downstream steps are able to be performed with fewer steps and reduced or no human intervention.

Embodiments of the disclosure overcome the disadvantages of conventional sample collection and testing workflows by providing systems, methods, and computer readable media for collecting information related to a sample collection event for pooled testing of a group of participants. Embodiments of the disclosure associate information related to the participants and information related to the sample containers collected at the sample collection event. In some embodiments, to facilitate pooled testing, a sample container contains specimens from two or more participants. Information related to the participants providing specimens in the pooled sample container and information related to the sample container identity is recorded at the sample collection event for use in subsequent testing of the pooled specimens. Embodiments of the disclosed systems may receive information related to the participants providing specimens and information related to the sample containers used at a sample collection event through a user interface in a mobile application or a web browser.

The embodiments described herein comprise systems and methods for improving the outcomes and operational efficiency of disease or pathogen testing programs. Rather than just testing members of an organization/entity, some embodiments comprise programs that register and include as testing program participants other people that have relationships or associations to the members. Detecting that, for example, a family member of a student is infected offers advance warning to the school that the student is at high risk and may become infected themselves soon. This extension of the testing program offers the opportunity for several types of mitigation measures, such as quarantining from an infected family member, staying home from school, or performing extra testing (e.g., “test-to-stay” model). These measures likely contribute to substantially reducing spread within the organization/entity.

In order to use these benefits of program extension and advance warning, it's important to know who the people are that are in close contact to organization/entity members, and to recruit, register, and track their participation and results. The embodiments described herein accomplish one or more such actions and improve their efficiency and accuracy of a testing program.

Some embodiments describe systems and methods to lower the barriers to registration of additional people in a testing program, and to easily receive information on identity and relationships or associations. Some of the embodiments comprise various modes of registering multiple individuals, including families, households, or association groups that may have exposure to one another. The registration process includes the collection of identifying information, such as name, date of birth, phone number and/or email, and may also include the collection of information pertaining to relationships or associations among the individuals or their relationship to an organization/entity.

Some of the embodiments describe a system that facilitates at-home pooled sample collection and at-home “on-the-fly” creation of pool manifest, which streamlines certain time-consuming aspects of lab processing in a testing program.

Further, some embodiments comprise systems and methods of displaying information associated with a disease testing program, possibly including user provided information from the registration process, onboarding information such as logins or signatures, testing participation information, test results, and “relevant information” related to actions, decisions, or status (e.g., quarantine period, return date). Displaying such relevant information in context of participants' relationships/associations may offer significant advantages in managing a testing program and achieving desired outcomes, which includes balancing the risk and potential exposure, keeping members out of quarantine (e.g., at school or at work), and program overhead and expense.

Some of the embodiments offer many significant health and economic benefits, which are often intertwined. First, early detection of infections reduces spread and results in fewer overall people infected, reducing the burden of disease in a population and improving an individual's chances to avoid immediate and long-term negative health outcomes. Secondly, early detection offers the opportunity for therapeutics to be administered earlier in the course of infection when they are more effective, hence the growth of “test-to-treat” programs supported by the U.S. federal government.

For organizations and entities, some of the embodiments not only enable improved health of its members, the embodiments may also help it to avoid staffing shortages and stay open, with the ensuing financial benefits. The advance warning of the potential infection of a member by detecting for example a family member as positive, gives the organization/entity the opportunity to adjust personnel and resource allocation to better adapt. The benefits to broader society, e.g., of staff outages from COVID at for example airlines and hospitals, have been substantial. Improvements in their testing programs, such as those implementing some of the embodiments, would do a great deal to reduce the economic drag of poorly mitigated spread.

Finally, the COVID pandemic has shown us how thoroughly unprepared we currently are for a dangerous new pathogen. Highly effective routine screening programs that are easy to administer by organizations/entities, easy to get started and participate in by the people being tested, flexible for both, and designed to stop disease spread, are a pillar of future pandemic preparedness.

When an organization/entity is able to gain visibility into not only the health of its members but also its members' household or exposure groups, it gains significant advance warning of infection threats to all its members. If infection is detected in individuals with a secondary degree of separation from the organization/entity, the organization/entity may protect its core members from disease outbreaks by instituting additional precautions where needed (extra testing, targeted quarantine). In essence, an organization/entity is then able to exercise an enterprise resource management strategy that best protects its members. Some of the included embodiments describe a program whereby this is possible.

School Example Including Some Embodiments

An example of a testing program that includes some embodiments is a school screening program. In this example, a school administrator sends emails out to the point-of-contact parent or guardian of every student and to every staff member. The email contains a basic description of how the program will work, an access code (a string of characters), and a URL link to a registration web form. The registration web form has fields for entry of names and contact information for various adults and minors, grouped by relationship. When one of the parents or guardians fills out the form for their entire family/household, accounts are created within a back-end database and information about the relationships of everyone registered through the form is stored.

The parents or guardians of the student can self-collect pooled samples at home, scan the pooled sample tube with a mobile app, and return the pooled sample to the school for processing. Test results from any individual or pooled test samples that include members of the family can be displayed and utilized in the context of the fact that they have relationships with each other and to the student, e.g., they all live together and if one is detected to have a highly transmissible virus, there is a high risk that others will become infected as well. The school administrators managing the program can use both the test results and the relationship information to make decisions about courses of action such as whether or not to require the student to stay home or test every day for some number of days before entering school, or to wear a mask.

When a family pool is detected as positive, it can be due to one or more of the family members being infected. The student of the school may not currently be infected or have a detectable level of virus, however detecting one of the parents or siblings as being positive provides advance warning that the student themselves may become infected, which could lead to the student spreading the infection at school. By testing families/households, the school garners an extra “layer” of protection. With the entire school participating in this family/household testing, it greatly reduces the chances of unknown infections among interacting students attending school. A twice per week program such as the example one using family/household testing may be more effective at stopping spread than one that tests every student daily. Further, if pooling of family/household samples is utilized, the cost and operational efficiency of the program is improved.

### BRIEF DESCRIPTION OF THE DRAWINGS

FIG. 1 illustrates an exemplary computer system that may execute an application used during a sample collection event or an exemplary computer system that may be used as a server communicating with an application used during a sample collection event.

FIG. 2 shows a design of an illustrative screen view of an exemplary application (FL App) wherein kits with containers of collected samples may have their status changed to “received” (aka the “intake” action) in preparation for subsequent processing, including, for example, testing at a lab.

FIG. 3 shows a design of an illustrative screen view of an exemplary application (FL App) wherein kits with containers of collected samples may have test results and notifications of those test results may be sent to participants.

FIG. 4 shows an exemplary flow chart of the steps performed on kits of collected samples, including steps carried out in an exemplary application (in FL App aka “virtual” steps) and steps in real life (aka “IRL”), the latter including steps that involve interactions with other users and testing program participants.

FIG. 5 shows a flow chart of the steps performed on kits of collected samples, including steps carried out in an exemplary application (in FL App aka “virtual” steps) and steps in real life (aka “IRL”), including steps that provide for the batching or grouping of kits together for subsequent processing.

FIG. 6 shows a schematic of an exemplary system including an application (e.g., FL App) executing on a mobile device with the application communicating with a cloud system (including storage and compute) using server code (e.g., FL Server Code) where the server code manages application information using a data object (e.g., FL Database).

FIGS. 7A-C show exemplary user interface screens for an application workflow to register an account.

FIGS. 8A-C show exemplary user interface screens for an application workflow to review and accept a consent review and acceptance workflow.

FIG. 9 shows an exemplary user interface screen for an application home screen.

FIG. 10 shows an exemplary user interface screen for an application participant personal information page.

FIGS. 11A-D show exemplary user interface screens for an application workflow to add a minor participant.

FIGS. 12A-E show exemplary user interface screens for an application workflow to collect manifest information.

FIGS. 13A, B show exemplary user interface screens for an application workflow to confirm sample collection.

FIG. 14A shows an exemplary results notification email.

FIG. 14B shows an exemplary user interface screen for an application results page.

FIGS. 15A-E show exemplary user interface screens for an application workflow to intake kits.

FIGS. 16A-D show exemplary user interface screens for an application workflow to enter results for tests conducted on kits.

FIGS. 17A, B show exemplary user interface screens for an application workflow to review kit test result history.

FIG. 18 shows exemplary workflows for setup and registration and test collections and results for an organization/entity.

FIG. 19 shows exemplary workflows for setup and registration and test collections and results for a relationship/association group.

FIG. 20 shows an exemplary system architecture implementing an embodiment of the invention.

FIGS. 21A, B show exemplary user interface screens for an application workflow to register a relationship/association group.

FIG. 22 shows an exemplary report of program status for a group of participants in a testing program.

FIG. 23 shows an exemplary report of test results for a group of participants in a testing program indexed by organization/entity member name.

FIG. 24 shows an exemplary report of family details for a group of participants in a testing program indexed by organization/entity member name.

FIG. 25 shows an exemplary report (in split view) of test results history.

FIG. 26 shows an exemplary report of program status and test results for a group of participants in a testing program aggregated by cohort (class).

### DETAILED DESCRIPTION

Some of the disclosed embodiments described herein distribute the workload of testing to the people who are themselves being tested, providing the tools for them to easily and accurately accomplish key steps. This shifts the burden from centralized chokepoints in the testing ecosystem out to the communities that need and want disease testing. By doing so, operational costs and complexity are greatly reduced which enables the frequency and turnaround time of testing to improve to the level that can greatly reduce the spread of the disease.

Some of the disclosure embodiments may be implemented using a mobile app running on a smartphone that is connected to server-side programs and storage on the cloud. In some embodiments, an app provides a key new functionality—the dynamic creation of the list of contributors to a pooled sample collection event that is linked to the pooled sample container, usually at the time and location of the physical collection event. This enables on-the-fly pooling in a traceable manner. Further, it enables pooling to be performed outside the lab such as at home, with members of the same household or a few households that form a “pod.” Some embodiments enable the tracking of individuals that are included in a pooled sample collection event and may be changed during collection (e.g., in a classroom based on the day's attendance) rather than being limited by a fixed list (e.g., list of students assigned by the school administration office). Some embodiments do not require scanning barcodes associated with individuals participating in the pool or entering the email addresses for participants during the sample collection event. Once a pool is created, the contributing participants may be stored as a default so the pool participants do not have to be re-entered for the same pool to be tested on a future date, while maintaining the capability to change the default set of participants.

The self-registration process permits users to sign up simply and quickly using a mobile app or using a browser and a web link. This registration may be part of the initiation of a testing program for a group such as a school, organization, or geographic population. In some embodiments, users may be required to enter an invitation code or to click an encoded hyperlink. In some embodiments, the sign-up process may be open to individuals without restrictions. In some embodiments, once a user creates an account, verifies their email address, and enters a password, they can login to the system, e.g., using an app on their smartphone or using a browser on any computer. In some embodiments, using the app or the browser, the user can find out information on how to get a clinical dx test or participate in a frequent screening program, if they are not part of one already. By accessing the system, e.g., from the app or browser, they may be able to further access information on testing, vaccination, restrictions or anything else related to public health or diseases (e.g., COVID). Key resources and information related to a specific testing program may also be available via the app or browser, including where to pick up, mail in, and drop off test kits. Instructions for sample collection and obtaining results may also be provided via the app or browser.

Some of the disclosed embodiments relate to the field of biological or chemical testing programs in which it is desirable to test for the presence, or in some cases quantification, of substances. Those substances may be pathogens (e.g., viruses or bacteria), chemicals, molecules, sequences of nucleic acids, proteins, or other materials (e.g., narcotics). The substances to be detected may be identified using a specimen obtained from people, in the form of biological specimens such as saliva, swab of a mucus membrane, blood, hair, etc., or using a specimen obtained from the environment (i.e., not directly from a person, e.g., a sample collected from a surface of a table, wall or floor). In some embodiments, a sample comprises specimens from only an individual person or an individual sampling device (such as a swab), referred to as “individual samples”. In some embodiments, a sample comprises specimens from more than one individual or more than one sampling device, mixed together into what are known as “pooled samples.” Individual samples can be combined at various points of a testing program to create a pooled sample. The term “samples” refers to whatever is tested, whether it is a specimen from a single individual person or single sampling device or combined specimens from one or more individuals, or more than one sampling device. As used herein, a “sample” is not limited to biological specimens from a single individual.

The testing program may utilize any combination of physical and digital infrastructure involved in the collection of samples. In some embodiments, a testing program may utilize a lab, field station, facility or other location in which at least a portion of the test procedures are carried out on the samples. Additionally, the testing program may utilize infrastructure involved in reporting results and other communications to the participants in the testing program. This reporting infrastructure is usually, though not necessarily, composed of digital technology including computer systems (e.g., servers in a datacenter or in the cloud, desktops/laptops used by the participants, mobile devices used by the participants) and computer programs (e.g., applications running on a server, application running on a desktop/laptop, application running on a mobile device).

In some embodiments, the digital infrastructure used in a testing program comprises information that may be kept in a data store (e.g., database, or the like). Manipulation of the information in the data store may be accomplished using an application using a user interface (UI), where the UI is presented to a user of a computer, mobile smartphone or other device when the application is executed. Information about participants is present in the data store, and, in some instances, present in database tables as records.

Other information in the digital infrastructure may include a variety of data about sample collection events (see, for example, Table 1), in some embodiments, including but not limited to the time and date of either a specific collection event from the sample collection event (e.g., collection of a specimen from a participant), entered concurrently with the physical collection of the specimen or within a certain time interval (e.g., within 10-120 minutes), or at a related time different from the physical collection event (e.g., when information associated with the sample collection event is received by the system). In some embodiments, the sample collection event data may include a manifest ID, see below. In some embodiments, the sample collection event data may include information related to a pooled sample container. In some embodiments, the sample collection event data may include information related to the location of the sample collection event (e.g., city and state).

TABLE 1

| Manifest ID | Sample Collection Event | PSample Container | Location | Date | Time |
|---|---|---|---|---|---|
| 4598231 | 2380943 | 3217859 | San Jose, CA | Dec. 13, 2020 | 8:45 AM |
| 4598231 | 2380949 | 7836201 | San Jose, CA | Dec. 17, 2020 | 8:45 AM |
| 4598232 | 2380956 | 8923561 | San Jose, CA | Dec. 19, 2020 | 8:45 AM |

Further digital information involved with the testing program may include a manifest, which is a term used for a group of participants. The manifest may comprise a list of one or more of names, email addresses, PII (personal identifying information), or de-identified IDs for participants in a pool. In some embodiments, the manifest is implemented as a table containing a manifest ID and one or more corresponding participant IDs (e.g., see Table 2, with manifest corresponding to manifest ID 4598231 including 4 participants). In some embodiments, a manifest ID may be used as a de-identified ID for a group of participants. In some embodiments, manifest information may also include a participant number. In some embodiments, the sample collection event, in addition to including collecting a pooled sample, includes collection of individual samples from the participants, see Table 3 showing sample container identifiers with individual samples from the participants.

TABLE 2

| Manifest ID | Participant Num | Participant ID |
|---|---|---|
| 4598231 | 1 | 8209479232 |
| 4598231 | 2 | 3141219390 |
| 4598231 | 3 | 2403424006 |
| 4598231 | 4 | 9624129348 |
| 4598232 | 1 | 8209479232 |
| 4598232 | 2 | 3141219390 |
| 4598232 | 3 | 2403424006 |
| 4598232 | 4 | 9624129348 |
| 4598232 | 5 | 5467382910 |

TABLE 3

| Sample Collection Event | Indiv ContainerID | Participant ID |
|---|---|---|
| 2380943 | 83843747347 | 8209479232 |
| 2380943 | 83843747440 | 3141219390 |
| 2380943 | 83843747474 | 2403424006 |
| 2380943 | 83843747498 | 9624129348 |
| 2380949 | 83843751170 | 8209479232 |
| 2380949 | 83843751182 | 3141219390 |
| 2380949 | 83843751250 | 2403424006 |
| 2380949 | 83843751315 | 9624129348 |
| 2380949 | 83843751402 | 5467382910 |

Yet further digital information involved with the testing program may include one or more of: 1) information related to of one or more participants who participate in a particular collection event (e.g., list of participants in the collection event), 2) records of containers used to contain samples (e.g., information related to container IDs), and 3) entries related to test results and the outcome of the process of tests.

In some embodiments, the participant starts by creating an account, though an account can also be created for them by someone else. There are various levels at which user involvement can be required during the account registration and personal information entry process. In one embodiment, a decentralized process involves a participant receiving an email, text message, or clicking a link on a website to begin the self-registration process. The self-registration process involves entering some basic information, typically first name, last name, and contact information, such as email or phone number. By default, the email address may be used as the username or the creation of a custom username can be permitted.

In some embodiments, the account registration process may require a verification step (e.g., verification of an email address provided during registration, verification of a phone number provided during verification—for example, by requiring entry of a code sent to the user's email address or phone number as SMS). In some embodiments, if a verification step is used, it may comprise sending an email to the email address provided. That account verification email may contain a link to click to take the user to a webpage or screen where the user can enter a password. Any suitable verification step may be used.

After account registration and password setup, the user may be directed to a login screen where they are prompted for their account credentials (username such as email address and password). Whether self-registration was used or not, upon first login the user (participant) may be asked to enter other information, potentially including demographic information, personal information, health information, or any other information (e.g., information that may be of use in the testing program). Some of this information may be utilized in reporting test results to various entities, in contact tracing, in statistical aggregation, or other uses.

In some embodiments, user roles may be assigned (e.g., at the time of account registration or upon completion of user profile), where those roles may have access to different digital functionality (e.g., permissions in a software application) or have requirements to manage processes in the real world (e.g., manage sample collection process). The role assigned to a user may be determined based upon information provided by the user, may be selected by the user or another user in the system, or may be set through any other means, (e.g., by an organization administering the testing program). Examples of roles may include one or more of: Participant, Minor, Staff, PI, and Administrator. See Application Workflow and User Set-Up in the Appendix Section for an exemplary description of a test flow that describes exemplary user actions based on exemplary user roles. In some embodiments, these roles may be stored as fields in a database, and then be utilized in determining access to various features in a software application.

In some embodiments, the “Sponsor” user type is responsible for the digital or physical sample collection process. In some embodiments, additional training may be required (or remain optional, if present) for the sponsor, and, in some embodiments, the training may be delivered via software, for example, via an app on a mobile device. In some embodiments, the training may be in one or more of a text format or a video format, and it may include a test process (e.g., online quiz) to confirm a certain level of familiarity with the role requirements and responsibilities. In some embodiments, the Sponsor role designation may include acknowledgement of having satisfied the role requirements. In some embodiments, one goal of utilizing a sponsor is to improve the reliability and safety of the sample collection process (e.g., oversee sample collection). In some embodiments, the sponsor ensures that the participants follow infection control procedures, in appropriate circumstances, such as physical distancing, wearing masks, and hand sanitation. An alternate embodiment to sponsor-driven collection is to have one or more participants in a sample collection event take actions that a sponsor may perform, such as implementing infection control procedures, maintaining integrity of the samples or sample container, scanning a barcode, entering data into a log, etc.

The designation of sponsors may be implemented in many different ways. In some embodiments, if a database system is used, sponsors may form a separate table, or they may be identified using a sponsor designation field in a general user table.

In some embodiments, the system maintains information related to sample collection events, see, for example, Table 1. The sample collection event information may include physical location information, for example, where the specimens were collected from the participants, or materials used during the sample collection event, e.g., barcoded, numbered or otherwise identifiable tubes (i.e., containers). In some embodiments, the sample collection event information may include the time and date or registration of the event, the location of the event, the participant(s) providing samples in the event, or information related to users who were submitting samples, supervising, or witnessing any portion of the sample collection event.

In some embodiments, associated with a sample collection event are one or more manifests that describe information related to the sample collection event. In some embodiments, the manifest includes information related to the participants, e.g., participants who submitted samples at the sample collection event, see, for example, Table 2. In some embodiments, the information related to the participants includes a non-private identifier (e.g., name, email address, phone number) for each participant. In some embodiments, the information related to the participants includes a private identifier (e.g., randomized ID) for each participant to maintain privacy of the participant. In some embodiments, the entries in the manifest may correspond to participants who are not registered users in the software or any other system used to record the manifest. Such participants may wish to be tested but also wish to maintain their anonymity or avoid registering. An entry may be any combination of characters, which can be predetermined or determined at the time of the collection event. In some embodiments, the manifest may be an ordered or unordered list. In some embodiments, some of the information in the manifest may be used with information related to sample containers (e.g., sample container ID) to identify the containers used during the sample collection event.

The system maintains information related to one or more sample containers. In some embodiments, the information related to the sample containers may be stored in a database, including as tables or fields. In some embodiments, the containers are test tubes with barcodes, human readable characters (such as text and numbers), or both barcodes and human readable characters. In some embodiments, the barcodes and characters may be related, such as the number for the barcode printed below it. In some embodiments, containers may be labeled on the caps of tubes, e.g., numbered 1 through 10.

In some embodiments, the entries in the manifest are in some way traceable without communication with the participants involved. An alternate embodiment includes verbal or private agreement by those present at the collection event regarding the actual identities of people contributing samples. Other embodiments permit completely or partially anonymous collection of samples, such as depositing unmarked tubes containing specimens into a bin, or the collection of pools where not all contributors of specimens in the pool are explicitly listed, say in a digital record.

Information for test results may also be stored in a database, or in any other record keeping system. These results can utilize typical terms such as positive, negative or invalid, or can include further qualitative or quantitative information related to the test. The test results can correspond to specific single instances of the test of a sample or to the aggregate determination (call) of the result for a sample, which may be a sample from a single individual or a pool.

In some embodiments, sample containers from the participants may be grouped and sub-grouped based on a variety of factors, and those various levels of grouping may allow for efficient batch processing in software implementations such as the FL app, see below in Appendix, or another implementation.

In some embodiments, a system for collecting information related to a sample collection event may comprise one or more applications executing on respective mobile devices in communication with one or more servers. In some embodiments, the system may receive, from a first application, a first set of information identifying the plurality of participants who are providing specimens during the sample collection event. In some embodiments, the first set of information may comprise a manifest ID which may be used to identify the plurality of participants.

In some embodiments, the first set of information may comprise a collection of unique identifiers that correspond to the plurality of participants. In some embodiments, the unique identifiers may match identifiers that were provided to each of the participants when they registered with the system (e.g., using the first application). In some embodiments, each participant may provide an identifier (e.g., name, email address, phone number) when registering with the system. In some embodiments, the system may assign a user record number to the registering participant with the user record number being associated with the participant identifier. In some embodiments, the system may assign a unique identifier for each participant based on the participant identifier or the participant user record number. In some embodiments, the first set of information may comprise a collection of the participant identifiers. In some embodiments, the system may provide each participant a unique identifier that is specific to the sample collection event.

In some embodiments, the system may receive, from a second application, a second set of information identifying one or more sample containers used to collect specimens at the sample collection event. In some embodiments, the second set of information may comprise an identifier for a sample container (e.g., from a barcode attached to the sample container, an identifier written on the sample container, etc.). In some embodiments, a system server may associate the first set of information and the second set of information to identify the set of participants who have provided a specimen to a sample container collected at the sample collection event. In some embodiments, the first application or the second application may associate the first set of information and the second set of information.

In some embodiments, the system may receive the first set of information and the second set of information from a single application executing on a device being used during the sample collection event. In some embodiments, the system may receive the first set of information from a first application executing on a first device, for example, to “check-in” participants at the sample collection event. The system may receive the second set of information from a second application executing on a second device (different from the first device), for example, co-located where the participants are providing specimens at the sample collection event. In some embodiments, the first application is the same as the second application. In some embodiments, the first application is different from the second application.

In some embodiments, a sample container may contain two specimens (e.g., two nasal swabs) in a single tube container. In some embodiments, the two specimens may be immersed in a liquid to preserve the integrity of the biological specimens. In some embodiments, the two specimens may be in contact with respective surfaces (e.g., inner surfaces) of the single tube container.

In some embodiments, the systems and methods comprise the collection of individual samples into separate containers, which may be later pooled together for testing. In some embodiments, one or more of the participants may use the FL app, or another disclosed embodiment, to create a manifest of participants at or around the time of collection. Individually collected samples (e.g., in sealed tube containers) may be bundled together (e.g., in a collection bag) for ease of transport and subsequent pooling. During the creation of the manifest, the participants may be changed and determined flexibly. Further, a de-identified piece of information, such as number or string, may be created and subsequently utilized during the processing of the individual or pooled samples. This de-identified piece of information may correspond to the barcode on a sample container, such as the sample container that is used to contain the pooled sample. Such embodiments provide individual samples and permit the assembly of pooled samples which can facilitate the determination of which individual or individuals are positive in a pool that tests positive, while using the systems and methods described herein—enabling real-time and distributed creation of information (e.g., manifests) for test programs. In some embodiments, the bundling of collection containers for subsequent pooling, manifest creation, and de-identifying information creation may comprise pooled samples to be pooled together again, thus creating pooled samples with even larger numbers of participants.

In some embodiments, the systems and methods involve integration with the LIMS/LIS (Lab Information Management System) of a testing lab. In some embodiments, information from the FL app or another implementation may be exported in different file formats to be imported into a LIMS/LIS, and vice-versa. Various features of LIMS/LIS systems may be added to the FL app or another implementation to expand or improve functionality. More specifically, barcode scanning, image processing, video and audio recording, and live streaming of video and audio may be added to the FL app or another implementation.

FIG. 2 shows a design of an illustrative screen view of an exemplary application wherein kits with containers of collected samples may have their status changed to “received” in preparation for subsequent processing, including, for example, testing at a lab. This screen includes the functionality of rejecting kits, notifying participants of rejected kits, and setting kits to the “processing” status to indicate the initiation of the testing step at a lab.

### Staff Receive View: Set Receive-Reject-Proecess

### Exemplary Staff Flow (FIG. 2)—Receive Kits:

1. Staff member gets kits from deposit site
2. Hit “Scan Barcodes” button 21 at top to turn on barcode scanner
3. Scan barcode of kit
4. Decides to reject or not, if so hits “Reject” button for that barcode and button lights up and says “Rejected”
5. If Rejected, then can also hit “Notify Rejected” button which lights up
6. Physically puts kits in OK or Reject bin as you go (make sure bins are labeled)
7. Takes bins to lab
8. Notify Rejected clears from this list and it goes in History

### Exemplary Staff Flow (FIG. 2)—Initiates Processing:

1. Staff member gets bin of OK kits
2. Decides which to run in lab in next assay batch (default all)
3. Sets up assay run in LIMS
4. For chosen barcodes, hits “Processing” button which lights up, or “Process ALL” 22

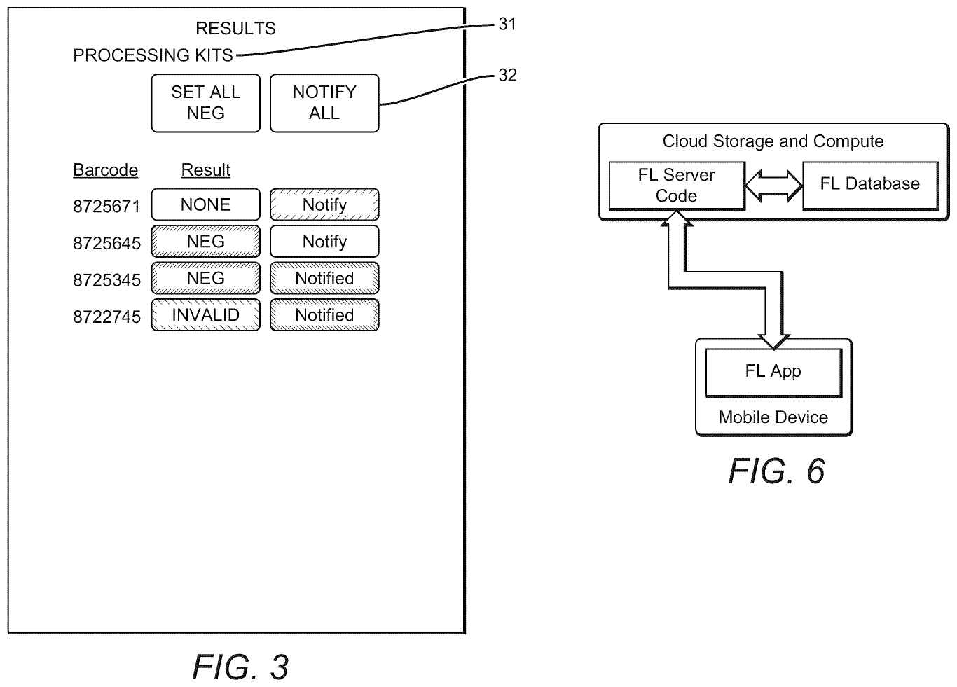

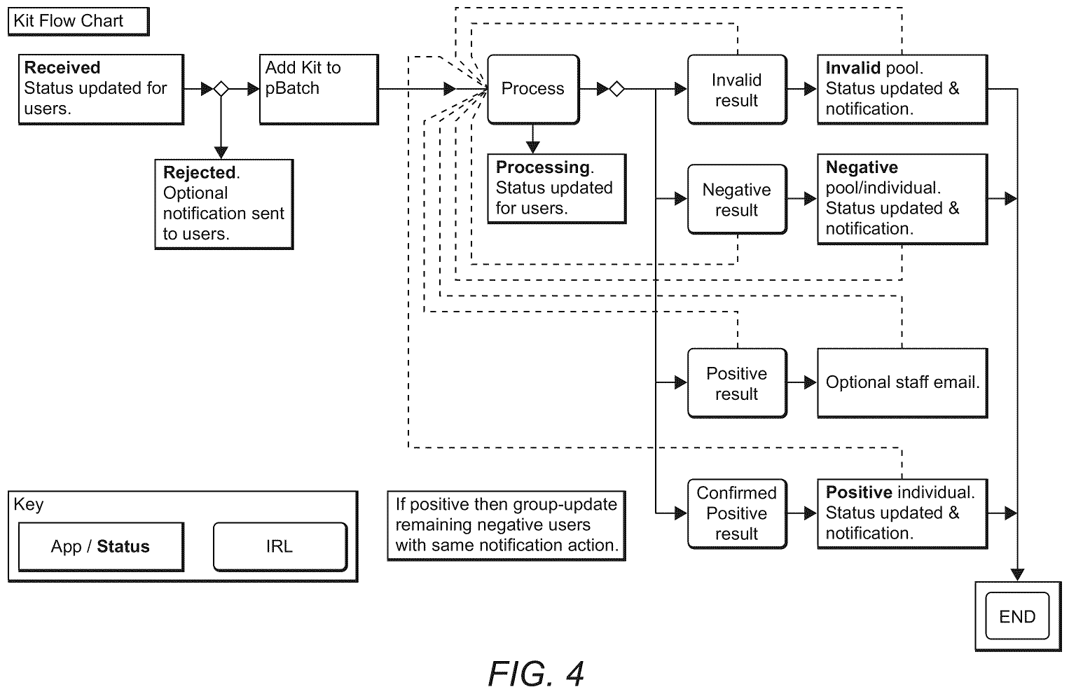

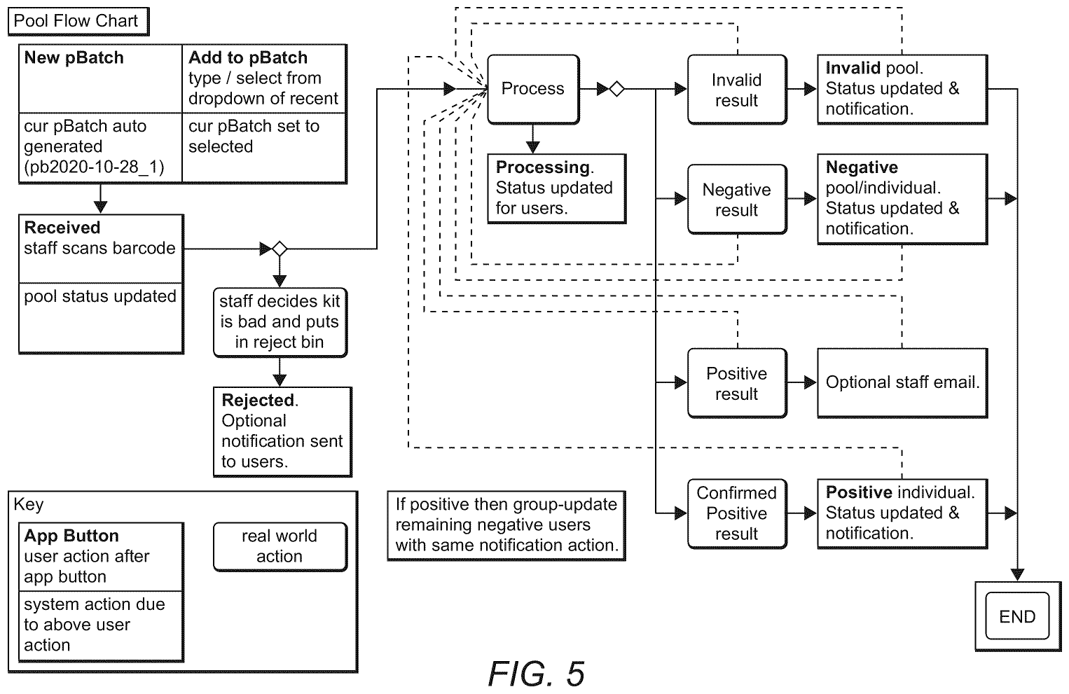

FIG. 3 shows a design of an illustrative screen view of an exemplary application wherein kits with containers of collected samples may have test results and notifications of those test results may be sent to participants.

### Staff Results View

### Exemplary Staff Flow (FIG. 3)—Results:

1. Staff member brings up Results page, typically after processing 31 is complete, including:
   a. Any reruns
   b. Any confirmation of pool positives
   c. Any runs of individual samples
   d. Any confirmation (w fluorim lamp or PCR)
2. Staff member enters results and chooses notifications 32 to be sent or not
3. Notes:
   a. Results entered in app are final results for the pool, not a result of a particular aliquot. Intermediate results, reruns, confirmations are all dealt with outside the app for now (in LIMS)
   b. NEG and INVALID generate specific messages that appear in the app.
   c. If participant has chosen, they will get notification that they have a new result and to check the app (don't display what the result is)
   d. Changing Result to POS sends an email to PI and anyone designated in group org that includes a specific message plus pool barcode and the manifest of participants in the pool (number, name, contact info)
   e. PI contacts lab staff to follow up on which individuals are positive and handles notification
   f. Only PI can select Notified on a POS, then POS becomes visible to participant
   g. Notify updates status that's visible to participant and sends notification according to participant preferences

In some embodiments, multiple people with a relationship or association to each other, participate in a testing program, such as a non-diagnostic disease surveillance program or a diagnostic testing program. A testing program may be administered in partnership with an organization/entity or entity, which may partially manage the testing. A “participant” in a testing program may comprise someone who contributes a sample only one time for a test, or many times for many tests, or they may be someone who registers for the program but does not actually contribute a sample for any test.

The registration process for a testing program may include various steps involving communications, permissions, and digital or analog systems. Some of the steps include opting into a testing program, granting permission for minors, providing a legal consent or waiver, creating digital accounts for individuals, adding information about individuals to a data structure (e.g., database or the like), and filling out a form on paper or via the web.

In some embodiments of the registration process, the organization/entity member or the other individual performing the registration, such as guardian for the organization/entity member, may register multiple people at once, including, for example, one or more additional members of their household. In such cases, the person performing the registration may include information about the other people for whom they are registering. Information provided in the registration process may comprise identifying information (such as first name, last name, date of birth, or identification number), contact information (such as email address or phone number), and relationship or association information.

In some embodiments, the relationship or association information may describe 1) family relationships, including that of a spouse, a guardian, a child, a dependent, a sibling, a parent, a grandparent, or any blood or legal relationship; 2) household relationships including people living together either full or part time, housemates, roommates, a care giver, a house guest, a domestic worker; or 3) associations including that of co-workers, friends, members of a social pod, or any association connected to people being within 10 feet of each other or occupying the same interior space. In some embodiments, people with the aforementioned family relationships, household relationships, or association comprise a “relationship/association group” for the purpose of a testing program. In some embodiments, a relationship/association group may register or be registered for a testing program at the same time, with a single registration form, or at various times with their relationship/association information included in the registration process.

In some embodiments, the person registering may register other people who are part of their exposure group (e.g., co-workers, friends, or members of a social pod). An “exposure group” is a term that refers to people who may potentially infect each other with a transmissible pathogen, and may comprises two or more individuals where each individual in the group: (1) has been within 10 m, 5 m, or 3 m of at least one other individual in the group, or (2) has occupied the same interior space of a structure as at least one other individual in the group. In some embodiments, proximity to another group member or sharing interior space with another group member for a period of 1 min, 5 min, 15 min, 30 min, or 1 hour or more may be required to satisfy the requirement of joining the exposure unit.

In some embodiments, the person registering may designate a participant's affiliation as a member of an organization/entity. The person registering may themselves be the member. A participant in a testing program may be designated to belong to a subgroup of an organization/entity (e.g., a school/university class, a department, a cohort, a building, or a designation of member type such as staff).

In some embodiments, an individual may belong to more than one household or exposure groups. In such cases, the person performing the registration may be asked to confirm the affiliation with both households (e.g., nannies or sitters who work for different households, individuals with stepfamily members, grandparents with grandchildren in different households). In some embodiments, adjustments may be made to the information regarding the people in a household after the registration event. In some embodiments, information associated with a participant (e.g., relationship, association) may be changed, e.g., by the participant or by another member in their relationship/association group.

In some embodiments, the registration event may cause the creation of multiple user accounts in a data structure, such as a database or the like. In some embodiments, registration is performed by the organization/entity through provisioning accounts through an interface, e.g., using an API, website portal, web form. In such cases, all or a significant amount of the information on multiple members of the organization/entity, as well as possibly some or all of additional participants with a relationship or association to members, is provided in advance and the organization/entity performs a bulk or batch registration, The process may comprise the upload of information directly to the data structure and/or create user accounts. For example, a school may already be in possession of family information for students and staff and accomplish batch registration by provisioning accounts for both the member students and staff and their families.

In some embodiments, the registration event may be self-directed by a person who belongs to the group being registered—resulting in the registration of a single group of individuals with relationships or associations. The organization/entity may not, through the actions of an administrator or employee, perform any registrations of testing program participants or create accounts, but may instead provide a web or paper form for participants to register themselves and any people with which they have relationships or associations. For example, a school administrator may just email a link to a website to the point-of-contact parent or guardian or each student, the email providing a website link to an online form to register some of all people in the family or household, where the relationship information of the family/household is stored as data and used in a testing program.

In some embodiments, the registration process may comprise a combination of an organization/entity provisioning accounts or performing the registration, and participants performing self-directed registration, possibly including additional people with whom they have relationships or association or confirming the existing information for participants. In some embodiments, the organization/entity may receive notifications on the registration status of organization/entity members and non-members through notifications or reports provided by using a website portal, an email, a text message, or another communications channel.

In some embodiments, testing programs may comprise individual testing or pooled testing, with pooled testing comprising samples from different people being combined together and tested simultaneously. In some embodiments, a registered organization/entity member may participate in pooled testing with other registered participants with whom they have relationships or associations. In some embodiments, a registered organization/entity member may be able to include an unregistered individual to their pooled collection via an alternate streamlined workflow, such as by entering their identifying information into a text field during the collection process. In such cases, the unregistered individual added to the pool may be asked to provide consent. In some embodiments, including an unregistered individual to a collection pool may trigger an “on-the-fly” registration workflow, whereby the individual is invited to register for a testing program and provide identification and relationship information.

In some embodiments, the registration process may be driven through a computerized system, such as a web application, a desktop application, a website, or a mobile application. The registration process may be performed manually, for example through paper forms, after which the information from the paper form may be further entered into a computerized or electronic system.

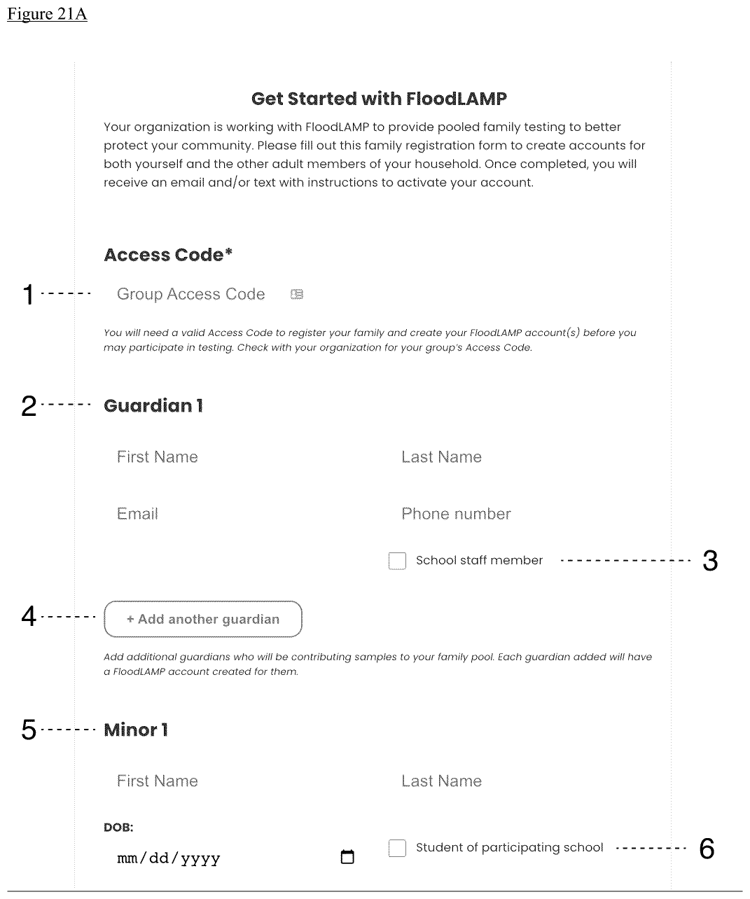

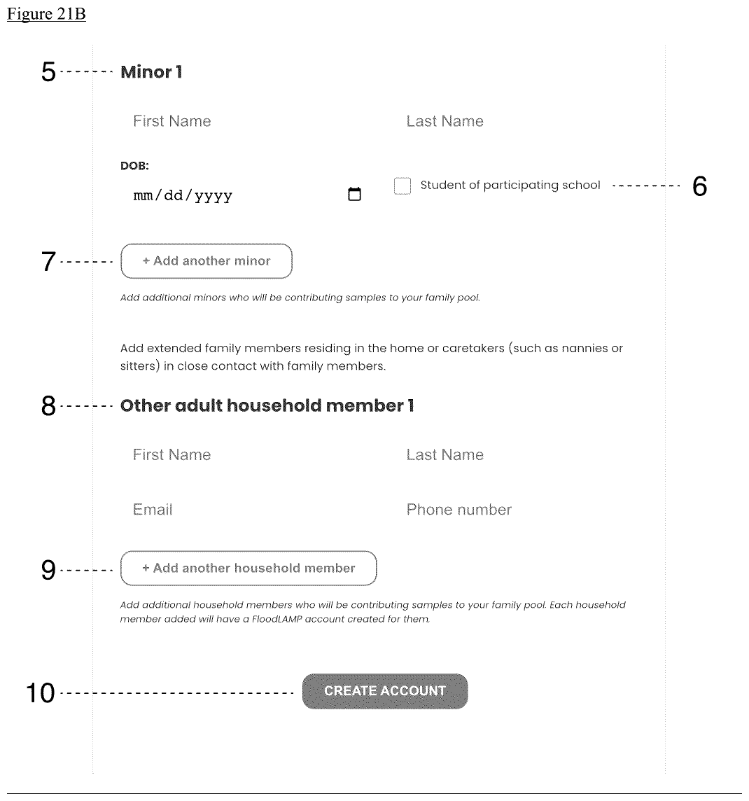

FIG. 21A/B illustrates an embodiment of a web-based registration form. An access code or token (1) may be included and can provide a layer of security for the organization/entity by validating a code or token that is provided only to members of the organization/entity or other people performing the registration. Additionally, the access code or token may ensure that any digital accounts utilized are created in the correct data structures or that appropriate settings are applied to the accounts registered to the individuals listed on the form or that digital accounts created in association with a given access code or token are associated with the correct organization/entity and, optionally, any subgroup such as class, pod, cohort, building, etc. Such settings may limit or grant the registered accounts' visibility or features (e.g., ability to view the directory of registered individuals, access to certain instructions or actions) as determined by the organization/entity. In some embodiments, testing program administrators of the organization/entity may configure the associated account settings and access prior to the registration process.

The access codes or tokens may be individualized and be created so as to be unique to a particular organization/entity member. In some embodiments, separate website URLs are used instead of access tokens. Additional security features such as encryption, authentication of identity through email hyperlinks, and two-factor authentication using cell phone numbers may be included.

Registration fields for capturing an individual's key information (2, 5, 8) may capture different user types (e.g., adult, minor), a member's affiliation with subgroups or divisions of the organization/entity (3, 6), or define relationships or association between the individuals being registered (e.g., parent, co-worker, child, sibling, extended family member). The relationships or associations may be predefined and for example be selected from a dropdown list, or may be unstructured and for example entered as a text. The fields for information capture may vary between organizations/entities in order to capture the relevant information per individual and/or the relationships/associations between the individuals registered in the form. In some embodiments, individual registration fields are dynamically added (4, 7, 9), and in some cases may correspond to the type of individual (e.g., guardian, minor, other adult).

In some embodiments, an action for triggering the creation of accounts for newly registered individuals (10) creates accounts for all the adults registered through the form or other registration process. In some embodiments, accounts may be created at this point for all individuals included in the registration process, including minors. In some embodiments, this form may be used to update information for one or more already registered individuals. In some embodiments, the account creation trigger initiates additional, separate workflows (e.g., account finalization, consent) that may be different for different individuals included in the registration process.

In some embodiments, a consent form for the policies of a testing program may integrated into the registration workflow. Guardians may provide consent for some or all of the minors being registered with a single signature, either physical or electronic signature. In some embodiments, members may not be fully enrolled unless everyone in their family, household, or association group has been included in the registration process and provided consent. In some embodiments, participants registered by someone else will be notified that they have been registered and prompted (e.g., by email, text, or call) to provide consent before they may be fully enrolled in a testing program.

In some embodiments, the registration process may add the names of the people being registered to a pooled collection manifest. In some embodiments, the manifest may include the relationship or association information between the individuals. In some embodiments, the relationship information is referenced with respect to the organization/entity member. Individuals added “on-the-fly” during a collection event may also be added to the pooled collection manifest.

In some embodiments, organization/entity members may perform collection events that include other organization/entity members. The registration process may include the control of approvals for the collection of pooled samples for a testing program. Information gathered during the registration process may inform pooled collection participant presets, which may be modified and adjusted later. For example, if a family is being registered, all the registered members of the family may be granted approval to perform a collection on each other but not anyone else.

In some embodiments, a member of the organization/entity or a person outside the organization/entity may be granted approval to collect samples from anyone in the organization/entity. For example, a teacher at a school may be granted approval to collect samples from any students or other participants.

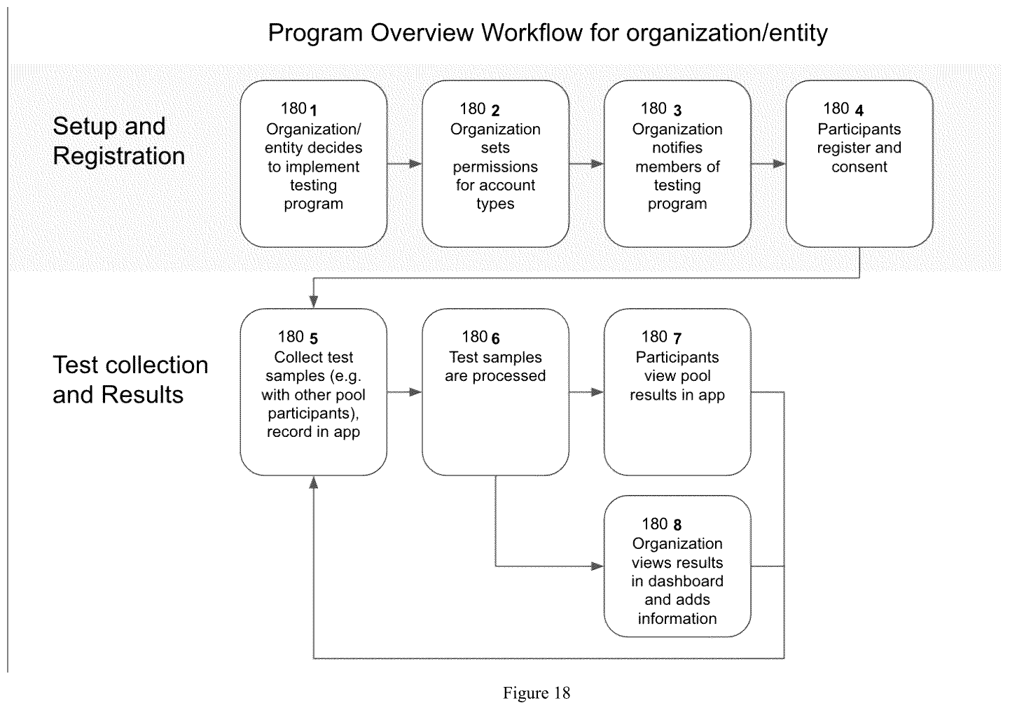

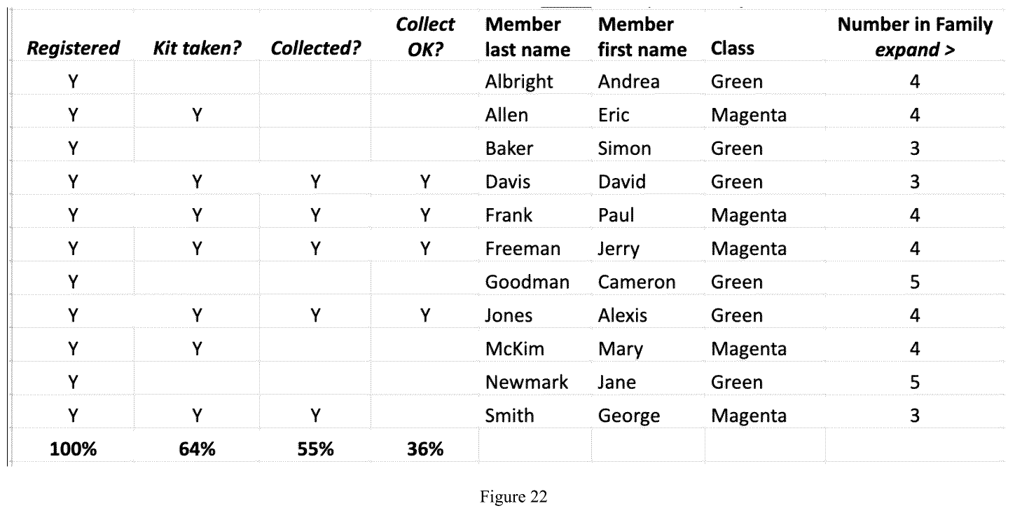

FIG. 18 illustrates an exemplary use case Program Overview Workflow for an organization/entity instituting testing for its members and relationship/association groups. The workflow is broken up into 2 main workstreams:
Setup and registration—these steps (1801-1804) cover the point from when an organization/entity decides to implement a testing program to when participants are registered;
Test collection and results—these steps (1805-1808) comprise a cycle that may repeat for repeated testing sessions of a testing program. In these steps, the participants collect pooled samples for the testing program, record the information about the collection participants, and track the results and other information in a dashboard, e.g., a view in an app or website. The organization/entity is able to access records for the complete testing program results, participation, and history—see, for example, FIG. 22.

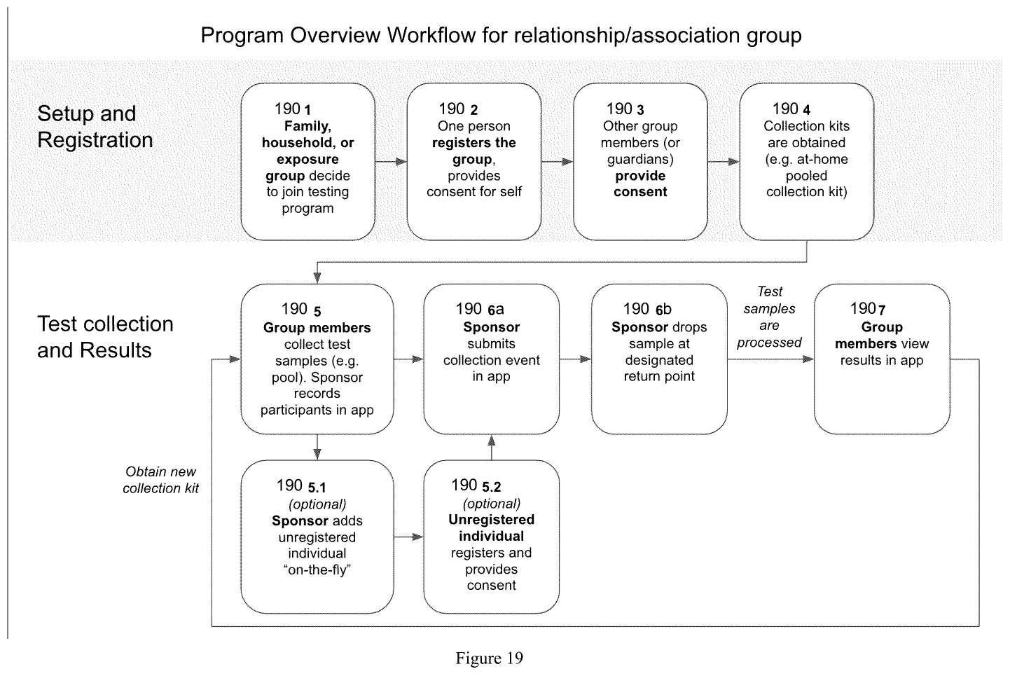

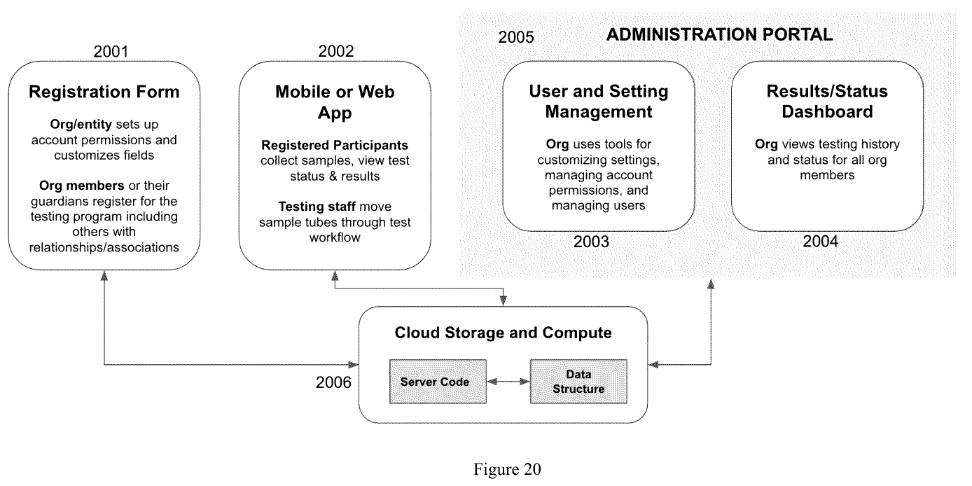

FIG. 19 illustrates an exemplary use case Program Overview Workflow for relationship/association group participating in a testing program, including a pooled testing program, that may not be implemented or actively managed through an organization/entity. The relationship/association group may be joining a program that is defined by a geographical area, or another attribute not associated with direct person-to-person interaction or potential disease exposure. The workflow is similar to that of FIG. 18. It is also broken up into 2 main workstreams:
Setup and registration—these steps (1901-1904) cover the point from when a relationship/association group decides to register the group into a testing program to when all the individuals of the group are registered.
Test collection and results—these steps (1905-1907) comprise a cycle that may repeat for repeated testing sessions of a program. In these steps, a participant (e.g., pool sponsor) manages the sample collection and listing of participants included in the pool in an app or site, and returns the sample for processing to a designated drop off site. The pool sponsor and all other registered group individuals are then able to track test results and history in the app or site. Optional steps 1905.1 and 1905.2 describe the process for previously unregistered users to be added to the workflow.

Reports

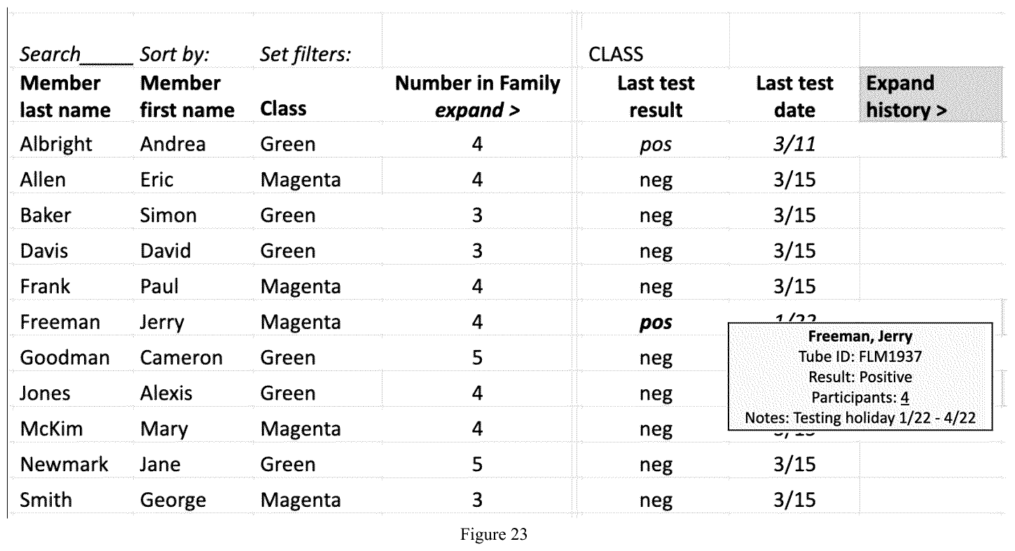

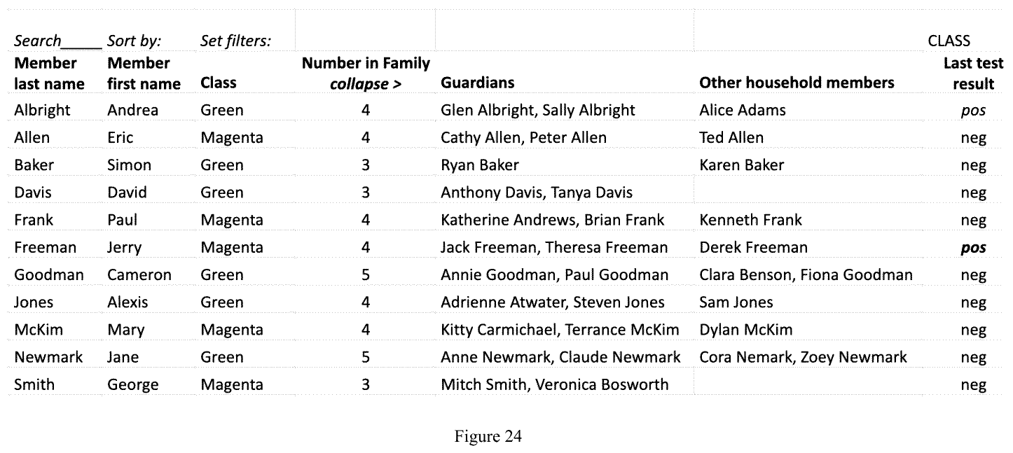

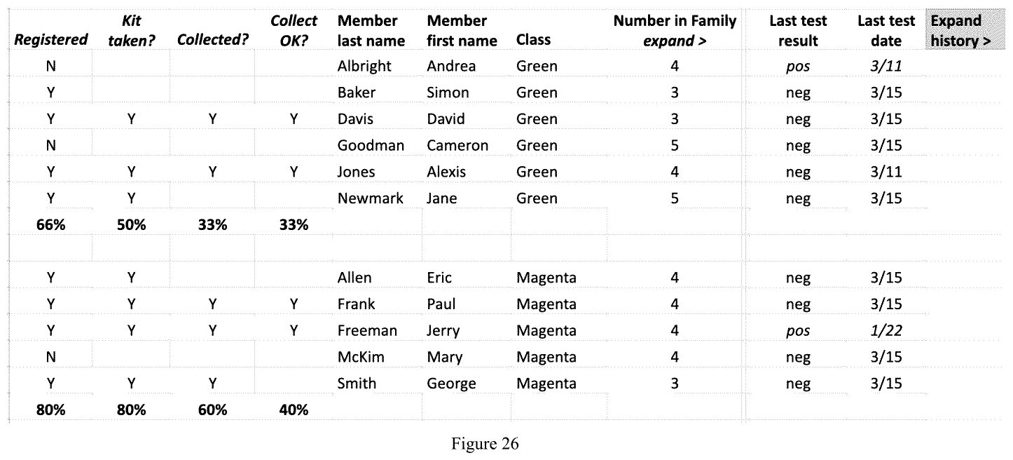

In some embodiments, information from the registration process may feed into a report, including a summary of testing program information (e.g., an information display, a “dashboard”), that acts as the front end for accessing testing program information, including, for example, the capability to manage aspects of a testing program. The information displayed may help to facilitate decisions related to one or more subgroups of an organization/entity to minimize outages resulting from spread of infection and manage access to on-site functions. In some embodiments, testing participation or results may be shown by a relationship/association group or by a group that pools their samples together or by both. In some embodiments, results are displayed indexed by organization/entity members (see, for example, FIG. 23), and the member's pooling group is also displayed in context (see, for example, FIG. 24). In some embodiments, the results may be indexed by members and also grouped by another division of the organization/entity, such as a cohort or department, for another option of the view of participation and test results (see, for example, FIG. 26).

In some embodiments, data may be tagged with metadata that allows for interactive hover or drill down states per data point or entity. In such views, additional information may be provided for participants (e.g., household members, cohort, most recent test, exposure status), test result (e.g., tube ID, pool participants, pool sponsor; see, for example, FIG. 23), or relationship/association group (e.g., number of members, exposure status, testing program participation summary).

In some embodiments, the display controls may be flexible and offer options for how to index the view, what information to show or hide, and how to group or sort the data. Some options for viewing the data may include but are not limited to:
Filtering views to an individual, pool, relationship/association group, whether organization/entity member or not, family, household, cohort, subgroup, result type, or time period;
Sorting views by name, result type, or status (e.g., quarantine period, testing holiday, exposure window);
Grouping views by cohort or subgroup, relationship type, or time periods;
Viewing testing program registration status (started, incomplete, or complete) (see, for example, FIG. 22)
Exportable views with options to anonymize data.

FIG. 22 shows an exemplary program workflow status for a plurality of participants (e.g., students) in two groups (e.g., classes identified by a color code). The status summary shows the progress for each participant through an exemplary workflow (e.g., including registration, collection of a test kit, collected sample returned for testing, and confirming the collected sample is acceptable). A summary of family information for each participant is also shown. Portions of the participant information from FIG. 22 is reproduced in FIGS. 23-26.

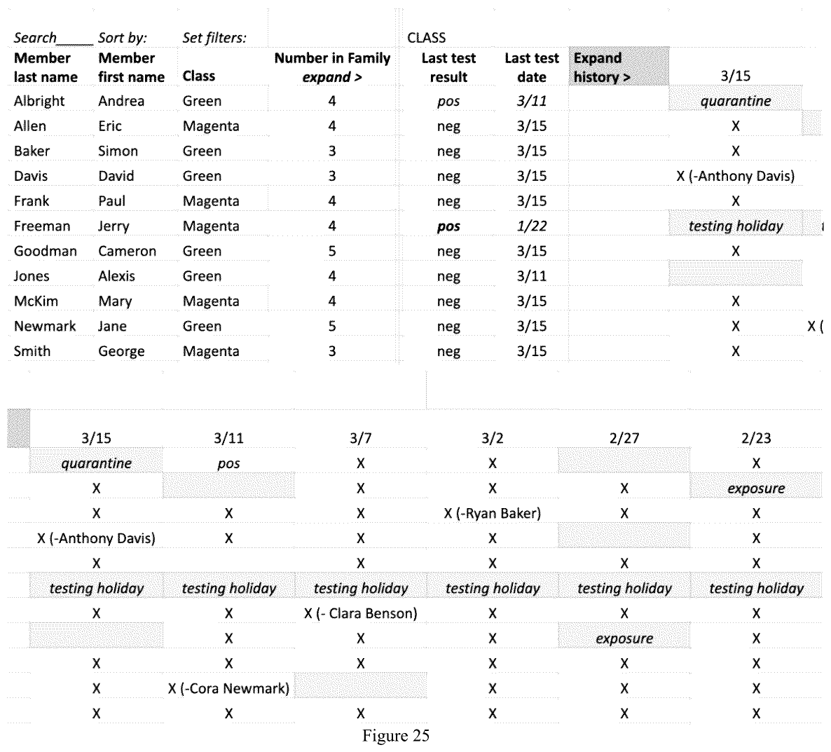

In some embodiments, testing results history may be visually laid out so that individuals' participation in a testing program over time is easily assessed (see, for example, FIG. 25). Such views may also be available at the pool level or any type of group or other division. In some embodiments, participants in a pool may vary over time and the participants in the pooled screening may be displayed over time.

In some embodiments, relevant information, including custom notes, may be added per participant, relationship/association group, or pool. The relevant information may relate to actions, decisions, or status that may concern modifications to some aspect of a participant interaction with the organization/entity or other people, or a change in mitigation measures. For example, they may include the time period to quarantine, a date of return or exit from quarantine, the duration of a testing “holiday” whereby testing is not required, a policy regarding returning to interaction or attendance with the organization/entity, mask wearing guidance or requirements, contact tracing and exposure notifications, testing frequency, the status of testing being voluntary vs mandatory. Relevant information may include any information related to or that may impact health status (e.g., disease exposure, exposure type, vaccination, symptoms, travel) or adherence to public health policies (e.g., quarantine windows, return-to-function timelines). In some embodiments, any of the relevant information or custom notes may be viewed in a calendar or timeline format (see, for example FIG. 25).

In some embodiments, exposure risk and time windows for quarantine and return (e.g., exit from quarantine) are automatically calculated and or recommended for individuals, relationship/association groups, or pools based on test results and administrator-controlled settings. Such settings may be customized to adjust the length of recommended quarantine or return periods to accommodate updates to information or broader recommendations (e.g., based on local public health, state, or CDC guidelines).

In some embodiments, for individuals with positive surveillance test results, follow-up diagnostic testing dates, test types, and results may be tracked and integrated into the dashboard display. In some embodiments, these results may be presented as a report. In some embodiments, multiple individuals or every participant in a pool may be included in the case report (e.g., a specific outbreak). In some embodiments, case report information may be anonymized for data export and sharing (e.g., with other entities associated with disease tracking).

This dashboard or information display may be available through a variety of media and formats. In some embodiments, the dashboard may be accessible through a web application or site, or a mobile app. In some embodiments, the dashboard may be an electronic document, such as a PDF or spreadsheet. In some embodiments, the dashboard may be a printed artifact, such as a paper report.

FIG. 20 illustrates an exemplary use case System Architecture that may be utilized to facilitate a testing program as described herein. The system may be deployed using cloud-based services 2006. Applications associated with the architecture may relate to one or more of an administrative portal or a standalone end user-facing application:
Registration Form 2001—may be customized by an organization/entity administrator, front end may be accessed by an organization/entity member or designated participant for registration process;
Mobile/Web App 2002—may be used by participants in the testing program for sample collection, which may include pooled collection, and for viewing test results or status; may be used by test processing staff to move sample tubes through the testing workflow to reflect status and results of tests.
Administration Portal 2005—may include User and Setting Management 2003, and a Results/Status Dashboard 2004. It may be accessed by an organization/entity administrator to view recent or historical testing data for registered participants, and may include relevant information that may be about modification/mitigation actions/decisions/status (e.g., quarantine periods).

In some embodiments, functionality of the Administrator Portal may also be accessible to participants, e.g., through the Mobile/Web App, and organization/entity administrators may access the Mobile/Web App. Roles for users of the system may be set to control access to various components and select views of data within the system, including reports (e.g., test result report, dashboard, group test summary report).

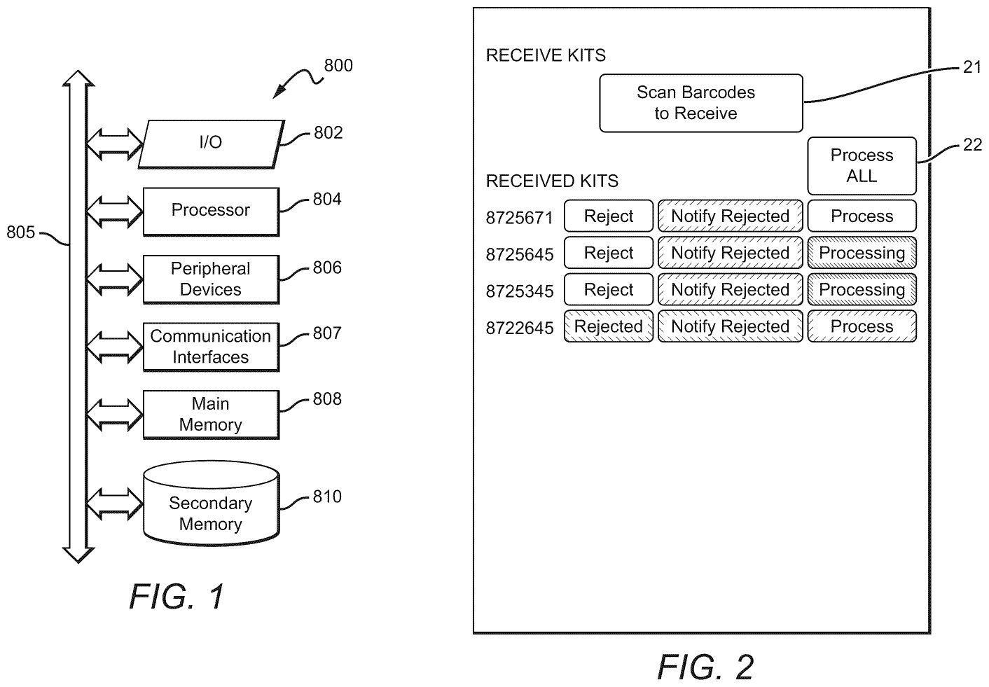

FIG. 1 illustrates an example of a computer system 800 that may be used to execute program code stored in a non-transitory computer readable medium (e.g., memory) in accordance with embodiments of the disclosure. The computer system includes an input/output subsystem 802, which may be used to interface with human users and/or other computer systems depending upon the application. The I/O subsystem 802 may include, e.g., a keyboard, mouse, graphical user interface, touchscreen, or other interfaces for input, and, e.g., an LED or other flat screen display, or other interfaces for output, including application program interfaces (APIs).

Program code may be stored in non-transitory computer-readable media such as persistent storage in secondary memory 810 or main memory 808 or both. Main memory 808 may include volatile memory such as random-access memory (RAM) or non-volatile memory such as read only memory (ROM), as well as different levels of cache memory for faster access to instructions and data. Secondary memory may include persistent storage such as solid-state drives, hard disk drives or optical disks. One or more processors 804 reads program code from one or more non-transitory media and executes the code to enable the computer system to accomplish the methods performed by the embodiments herein. Those skilled in the art will understand that the processor(s) may ingest source code and interpret or compile the source code into machine code that is understandable at the hardware gate level of the processor(s) 804. The processor(s) 804 may include dedicated processors such as microcontrollers running firmware. The processor(s) 804 may include specialized processing units (e.g., GPUs) for handling computationally intensive tasks.

The processor(s) 804 may communicate with external networks via one or more communications interfaces 807, such as a network interface card, WiFi transceiver, etc. A bus 805 communicatively couples the I/O subsystem 802, the processor(s) 804, peripheral devices 806, communications interfaces 807, memory 808, and persistent storage 810. Embodiments of the disclosure are not limited to this representative architecture. Alternative embodiments may employ different arrangements and types of components, e.g., separate buses for input-output components and memory subsystems. Elements of embodiments of the disclosure, such as one or more servers (e.g., in the cloud) communicating with an app, may be implemented with at least some of the components (e.g., processor 804, memory 808, communication interfaces 807) of a computer system like that of computer system 800.

Those skilled in the art will understand that some or all of the elements of embodiments of the disclosure, and their accompanying operations, may be implemented wholly or partially by one or more computer systems including one or more processors and one or more memory systems like those of computer system 800. Some elements and functionality may be implemented locally and others may be implemented in a distributed fashion over a network through different servers, e.g., in client-server fashion, for example.

In some embodiments, an application may be an application executing in a mobile operating system (e.g., iOS from Apple, Android from Google). In some embodiments, an application may be a desktop application designed to run in an operating system such as macOS from Apple, Windows 10 or 11 from Microsoft, ChromeOS from Google, etc. In some embodiments, a browser may be a browser such as Chrome from Google, Edge from Microsoft, Firefox from Mozilla, etc. or a browser extension designed to run on such a browser in an operating system (e.g., macOS, Windows 10 or 11, ChromeOS, etc.). Any of these executables may run on a computer system such as computer system 800. In some embodiments, a system may comprise one or more mobile applications executing on respective mobile devices and one or more server applications executing on one or more servers (e.g., in the cloud—Microsoft Azure, Amazon AWS, Google Cloud Platform, etc.).

Those skilled in the art will recognize that, in some embodiments, some of the operations described herein (e.g., acquiring a specimen from a participant) that do not involve data processing may be performed by human implementation, or through a combination of automated and manual means.

Although the disclosure may not expressly disclose that some embodiments or features described herein may be combined with other embodiments or features described herein, this disclosure should be read to describe any such combinations that would be practicable by one of ordinary skill in the art. The user of “or” in this disclosure should be understood to mean non-exclusive or, i.e., “and/or,” unless otherwise indicated herein.

In the embodiments or claims below, an embodiment or claim reciting “any one of embodiments/claims X-Y” shall refer to any one of embodiments or claims from embodiment/claim X and ending with embodiment/claim Y (inclusive). For example, “The system of any one of claims 7-11” refers to the system of any one of claims 7, 8, 9, 10, and 11.

Each embodiment below corresponds to one or more embodiments of the disclosure. Unless indicated otherwise, dependencies below refer back to embodiments within the same set (e.g., Embodiment Set A, Embodiment Set B, Embodiment Set C, . . . ).

In embodiments described in combination with one or more parent embodiments, an embodiment element having a name that matches a corresponding, identically-named element in any of the one or more parent embodiments correspond to the same element—for example, in Embodiment Set A, in embodiment 17 based on embodiment 16 based on embodiment 12, the “the association” of embodiment 17 corresponds to “an association” of embodiment 16 and “an association” of embodiment 12.

Embodiments that are incomplete or not practicable as a result of nested use of multiple dependent embodiments may be ignored. For example, in Embodiment Set A, embodiment 26 recites “the entity.” However, the set of embodiments 26 depending on embodiment 19 depending on any one of embodiments 1-10 may be ignored because embodiments 1-10, 19 do not recite “entity.”

### APPENDIX: APPLICATION WORKFLOW AND USER SET-UP

User Roles

1) Users of the application that may participate in a sample collection event—one or more types of individuals that have varying permissions in using the application based on their role; user roles may include:
Participant—a person who is being tested/screened; a Participant may have limited permissions in the application (e.g., permission to create a account/profile, permission to edit their account profile, provide consent, etc.); the application may require consent from or on behalf of a Participant (e.g., if minor, see below)
Sponsor—an end user that initiates a sample collection event; a Sponsor may have elevated permissions in the application compared to a Participant (e.g., to create and manage/edit manifests); a Sponsor may be responsible for proper sample collection (e.g., participant exposure safety, sample integrity, sample container tracking), creation of sample manifest, etc.; a Sponsor may also be a Participant.
Minor—an end user who does not have authorization to provide consent on their own behalf (e.g., if they are a minor, under the age of 18, lacking capacity to provide legal consent, etc.); a separate consent form may be provided by another Participant (e.g., parent/guardian for the minor); a Minor may provide a sample during a sample collection event; a Minor may be restricted to participate only in a sample collection event that also includes the Participant who provided consent on their behalf or another Sponsor who is identified to manage sample collection for the minor (e.g., a teacher approved by a Participant parent of the Minor).

2) Staff—a user type that may have permissions associated with actions involving samples after they are collected by Users or actions involving other Users (e.g., user account creation, etc.); Staff may have permission to receive a sample (e.g., take possession of collected samples), process a sample (e.g., perform one or more tests on the sample in the lab or field), communicate results related to a sample, delete a sample (e.g., if the sample is discovered to be contaminated), update/correct a sample manifest (e.g., if an error is identified); Staff may also have one or more permissions attributed to any other User type.

Other User Roles:

PI—a user that has permission to receive test results related to one or more sample events; a PI may receive an email concerning a positive result for a pool, (e.g., including the manifest and contact information of pool members). A PI may receive other sensitive information, or status reports, data summaries, etc. The PI may have access to personal identifying information for participants, such as the names and contact info. Other user roles may have access to de-identified results (e.g., to monitor positivity statistics, etc.). PI may also have one or more permissions attributed to any other User type.

Administrator—permission to change/manage application configuration (e.g., database reference/structure/location, system configuration, other application backend configuration); Administrators may have permissions to: modify roles, etc. Administrator may also have one or more permissions attributed to any other User type.

The exemplary operations/workflows, shown below, will be labeled as “Participant” when they apply to all Participants or Staff, (though technically we could adjust the staff role to not have the same permissions as users in that group/data silo) and labeled as “Staff” when they apply only to Staff and not to non-Staff Participants.

Onboarding (All)

Account Registration (All)

Users may sign up for an account within the application through an onboarding flow. Successful account creation is messaged to the user.

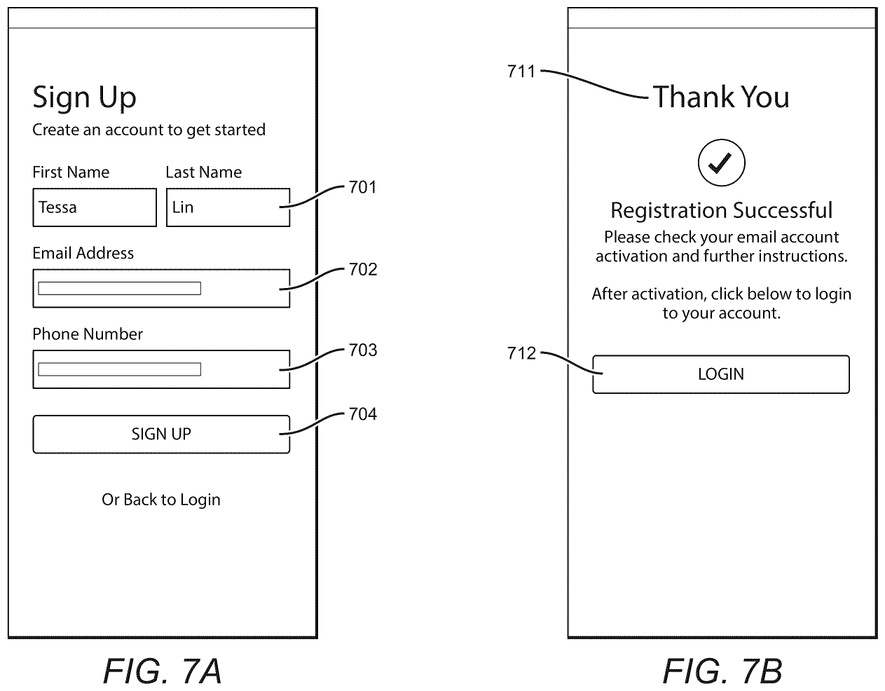

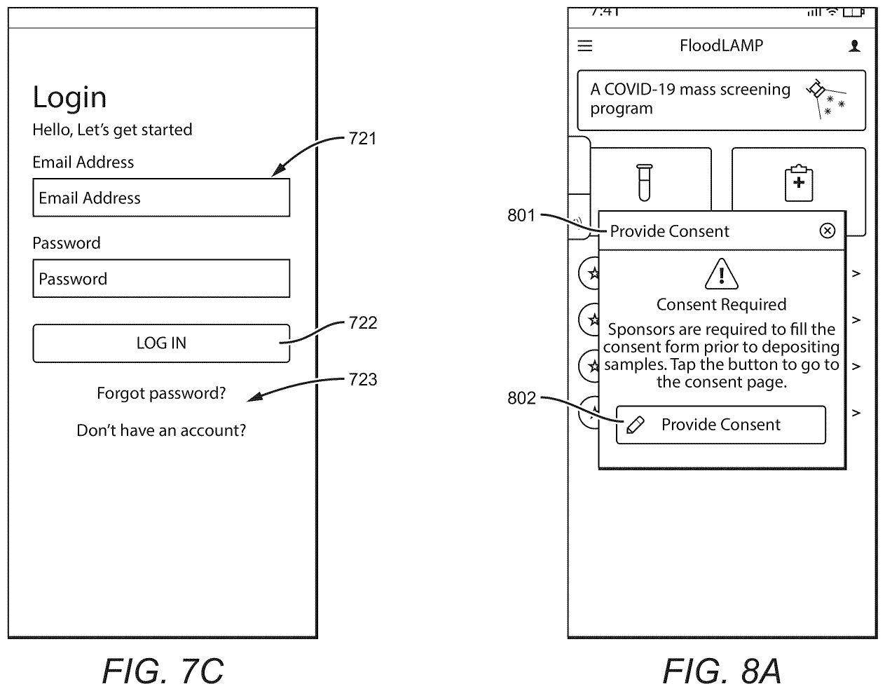

FIG. 7A
1. Name fields 701—prospective participants must provide First and Last names.
2. Email address field 702
3. Phone number field 703
4. Sign Up button 704—submits information for registration

FIG. 7B
1. Registration feedback screen 711
2. Login button 712—navigates to login screen

FIG. 7C
1. Login credentials 721—email address and password
2. Login button 722—navigates to the Home Screen
3. Links for password reset and account creation screens 723

Consent (Participant)

First time Participant users are required to review and sign a consent form (e.g., FloodLAMP consent form) before accessing any features of the application. The Participant may not advance in the flow without signing the consent.

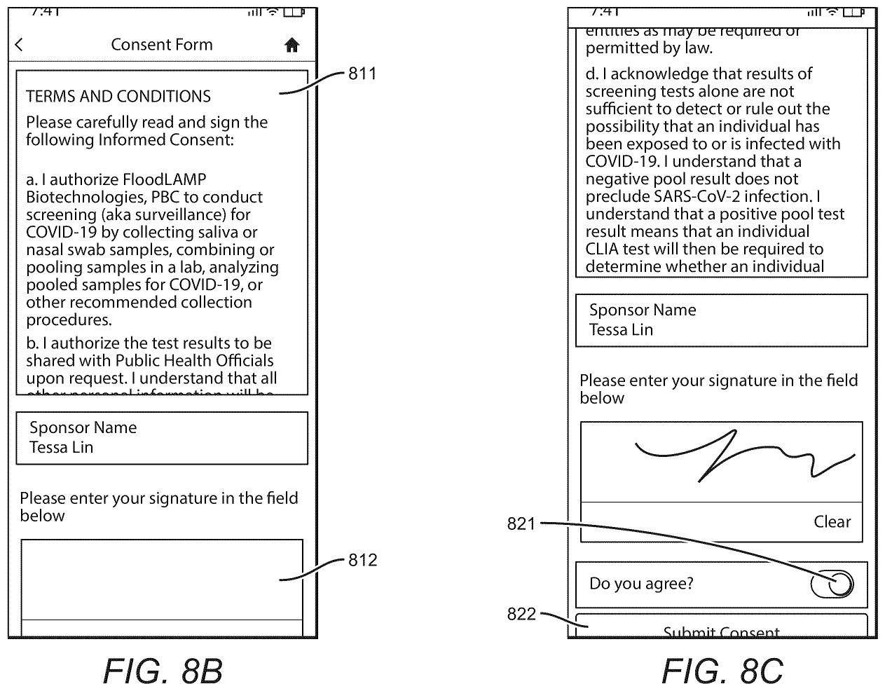

FIG. 8A
1. Consent required popup 801—first time use of the application requires signing the consent in order to access all other functionality.
2. Provide Consent button 802—triggers the Consent form

FIG. 8B
1. Consent form 811
2. Digital signature field 812

FIG. 8C
1. Consent toggle 821—this must be activated in order to proceed
2. Submit Consent button 822—this is disabled until both the signature field and consent toggle have been filled/activated. Tapping completes the consent flow.

Home Screen (Participant)

Participants land on the home screen after login. This screen offers direct pathways to all core user workflows.

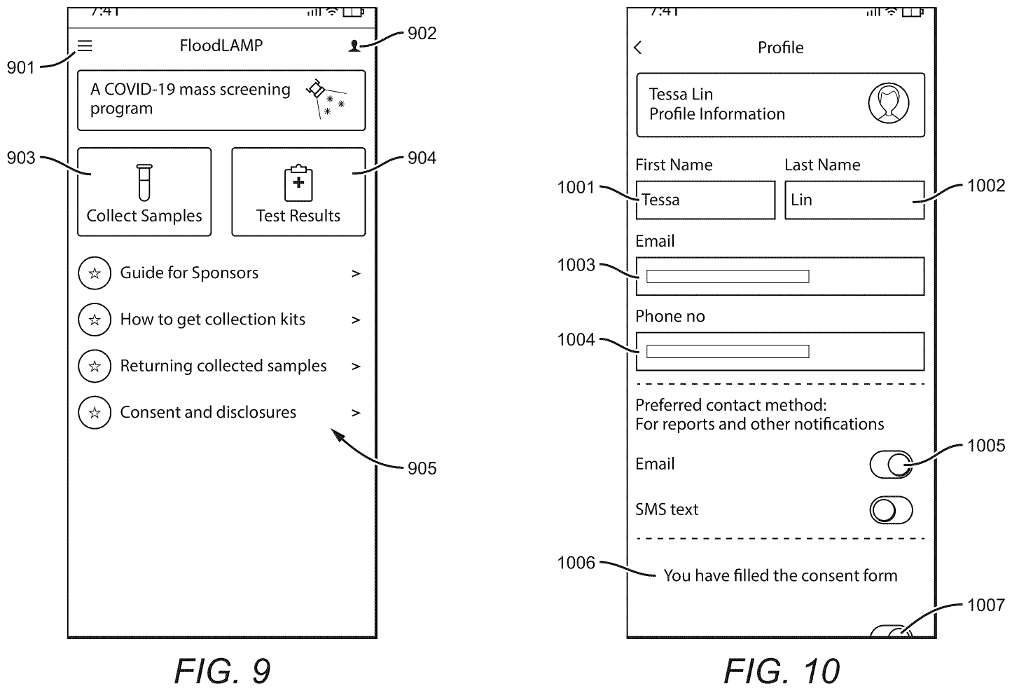

FIG. 9
1. “Hamburger” Menu 901
2. Profile link 902—navigates to user profile, see Home Profile Link section of this document for user flows
3. “Collect Samples” action button 903—see Collect Samples section of this document for user flows
4. “Test Results” action button 904—see Test Results and History section of this document for user flows
5. Informational links 905:
   Guide for Sponsors—offers instructions for sponsor
   How to get collection kits
   Returning collected samples
   Consent and disclosures

Home Hamburger Menu (Participant)
Home
Profile
Guide for Sponsors
FloodLAMP website
Switch to Staff Role
Logout

Home Profile Link (Participant)

Personal Information

Profile page stores and displays participant information provided upon registration as well as user settings and ability to manage minors associated with the participant account.

FIG. 10
1. First name 1001
2. Last name 1002
3. Email 1003
4. Phone Number 1004
5. Preferred contact method (toggles for email and SMS txt) 1005
6. Consent form acknowledgement (text display only) 1006
7. Children/Minor link (toggle and entry form if ON) 1007—see Adding Minors section of this document for flow.

Adding Minors (by Participant)

A registered Participant may add minors/dependents from their profile settings. Each added minor/dependent may require the Participant to sign a respective consent form.

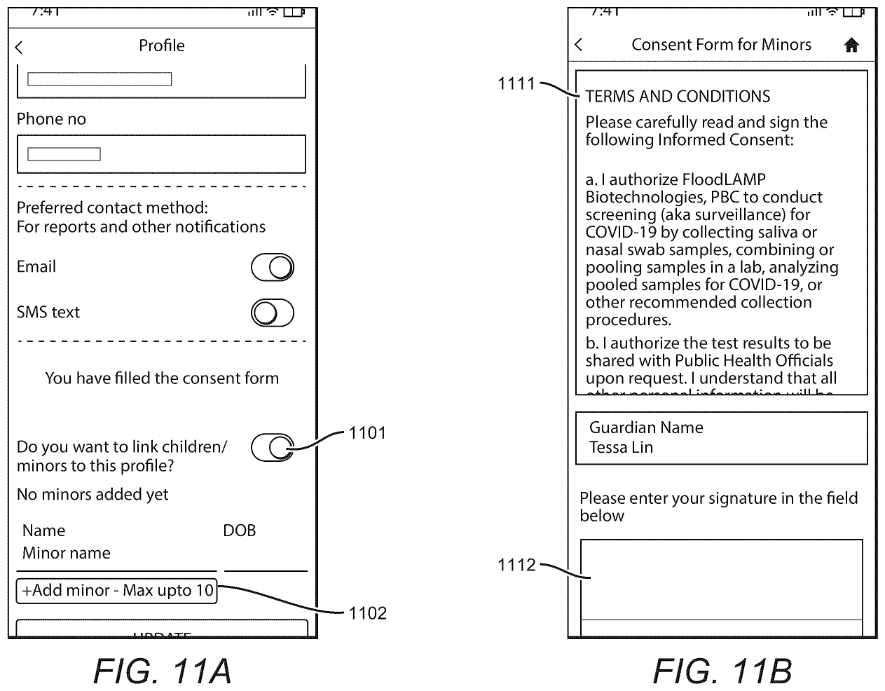

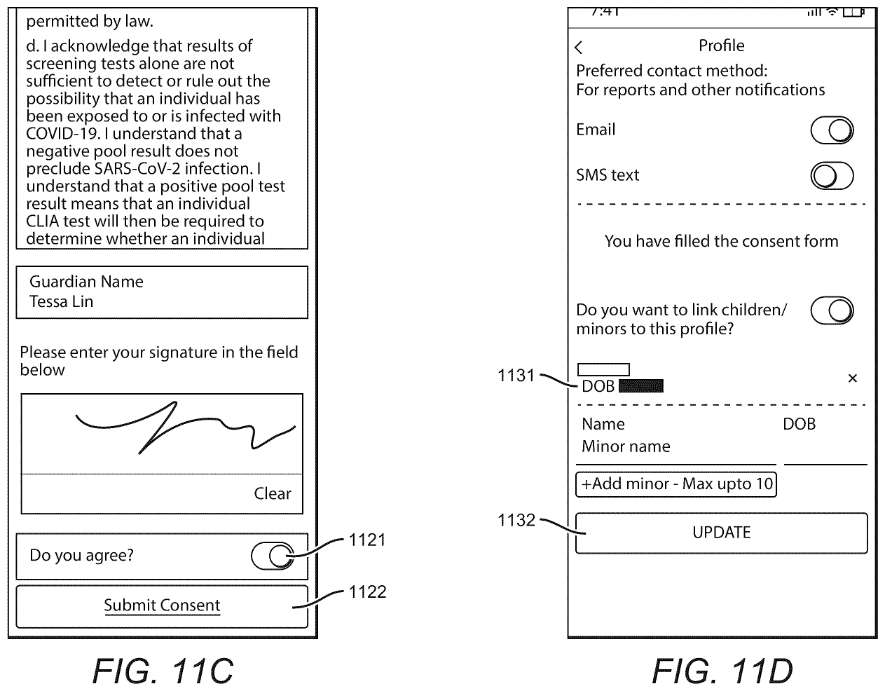

FIG. 11A
1. Add minor toggle 1101 activated, exposes field for adding minor name and DOB
2. Add Minor button 1102—initiates workflow for adding minor, triggers consent form for guardians.

FIG. 11B
1. Consent form 1111—scrolls within a contained field
2. Signature field 1112—field for participant's digital signature

FIG. 11C
1. Consent toggle 1121—must be activated for Submit Consent button to be enabled
2. Submit Consent button 1122—finalizes the addition of minor to participant profile.

FIG. 11D
1. Successfully added minor name and DOB displayed 1131. Red X enables deletion of minor.
2. Update button 1132—saves changes to the participant profile.

Collect Samples aka Collect (Participant)

A participant may begin a collection event by selecting the “Collect Samples” button on the Home screen. Within the “Collect” flow, the participant lists all the Participants or Minors in the pool for the designated Collection event. Minors may only be added if they are associated with the participant's profile. The pooled samples may be collected in a barcoded tube and the barcode may be either scanned or manually entered. The participant may be prompted to proceed and may perform final checks prior to submitting their pooled sample information.

Collect Manifest Screen

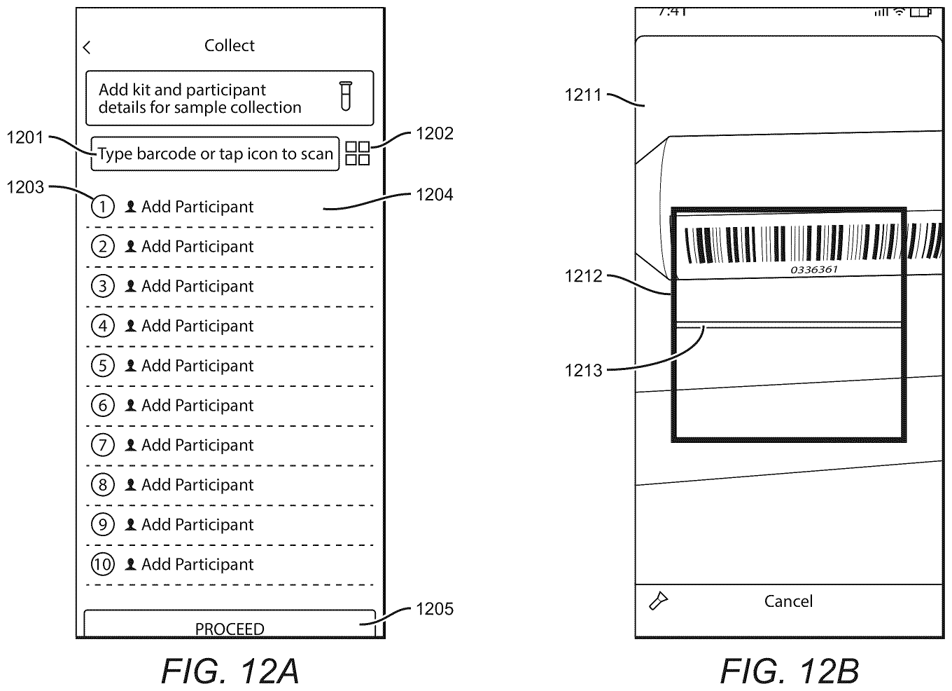

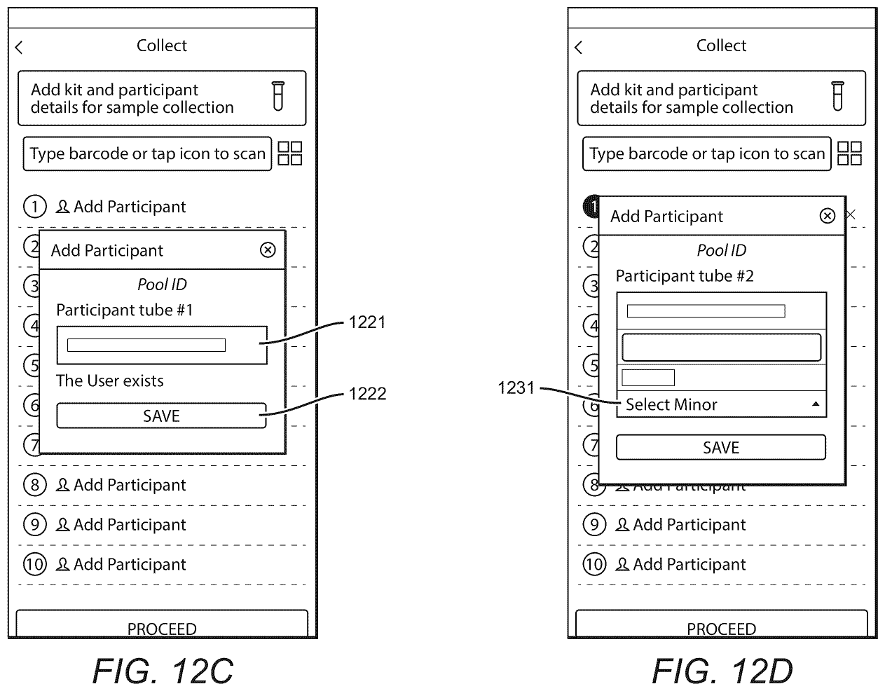

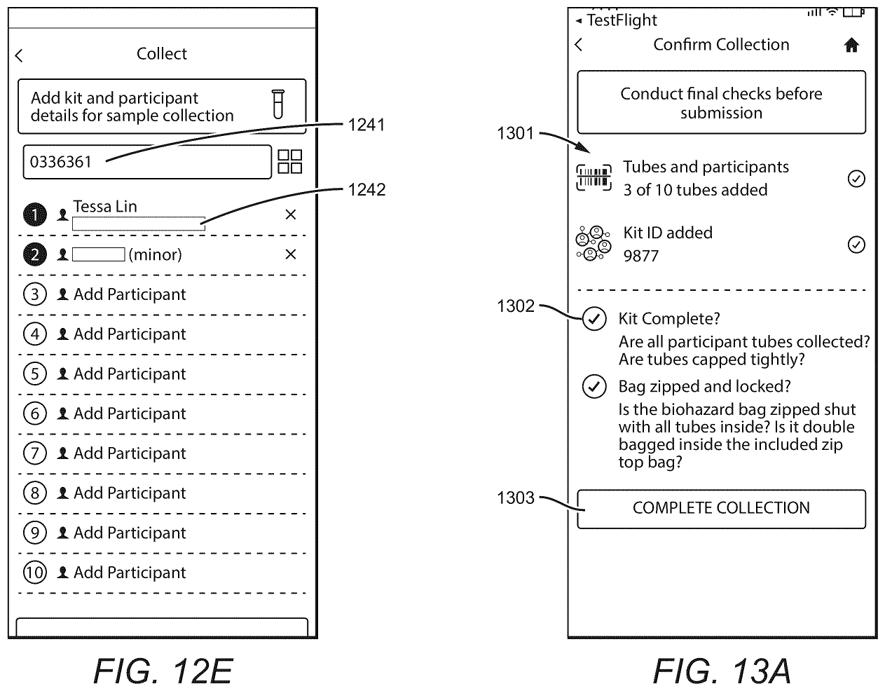

FIG. 12A
1. barcode or container identifier entry box 1201—the user can manually type into this field
2. barcode scanning initiation button 1202—triggers the barcode scanning tool, turning on the smartphone camera and opening the view shown in Fig CMS3
3. participant number id 1203—may correspond to the number on a container or material (such as a swab) used for individual collection, or simultaneous individual and pooled collection. The number can be printed or written in advance or at the time of a collection event.
4. participant name fields 1204—participant names appear in these fields after they have been added. Tapping the field triggers popover for participant information input.
5. proceed button 1205—initiates collection submission flow. Is disabled until both participants and barcode are added. See Collect Confirmation Screen section of this document for user flows.

FIG. 12B
1. camera view 1211
2. barcode target window 1212
3. barcode target line 1213

FIG. 12C
1. Participant identification field 1221—text input field where sponsor may enter the email address or username of the participant contributing samples to the collection event. In a preferred embodiment, email addresses are used and stored as defaults for subsequent collections.
2. Save button 1222—records the participant information for the collection event.

FIG. 12D
1. Select minor dropdown 1231—if a participant has added minors to their user profile, saved minor names appear in drop down selections.

FIG. 12E
1. Barcode added 1241
2. Participant information completed 1242—names and email addresses (blacked out) are visible. Participant names may be removed by tapping red X.

Collect Confirmation Screen

Participants must complete final checks in a confirmation flow prior to submitting their sample to the system for collection.

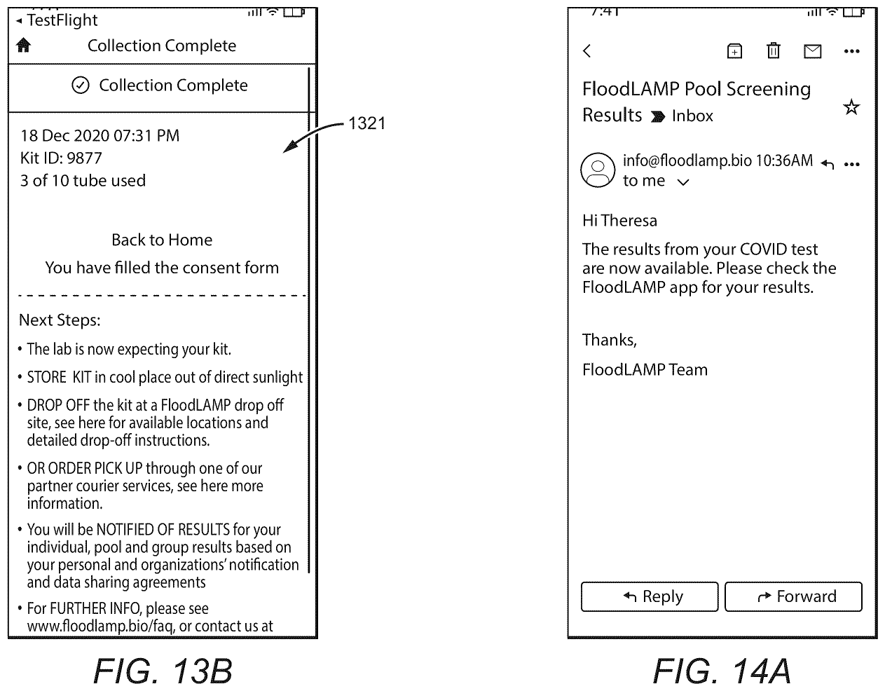

FIG. 13A
1. Submitted information is displayed in this section 1301 (tubes/participants and Kit ID)
2. Final checks radio buttons 1302 are unfilled until tapped. Tapping toggles their state.
3. Complete Collection button 1303—submits the collection. The button is disabled until the checks are completed.

FIG. 13B
1. Collection Complete acknowledgement Screen 1321

Test Results and History (Participant)

Participants may receive a notification with an indication of their test results (e.g., test result is available, test result is positive, test result is negative, etc.)—notification may be provided in the application (e.g., Apple Notification in iOS) or by phone/voicemail, SMS or email depending on their profile settings. The non-App notification may prompt them to visit the app and view their test result, as is best practice for mobile apps. If there is a problem with their sample (spilled, destroyed), they may be notified that their kit is rejected. Participants may view test results status of “In Process” and previously submitted collection events within their History screen.

Negative results may be reported to participants and positive results may trigger an email to be sent to the PI, with a list of the manifest and other information on the sample/pool/barcode. The PI may approve a positive result to be transmitted to one or more participant(s) via the notification process.

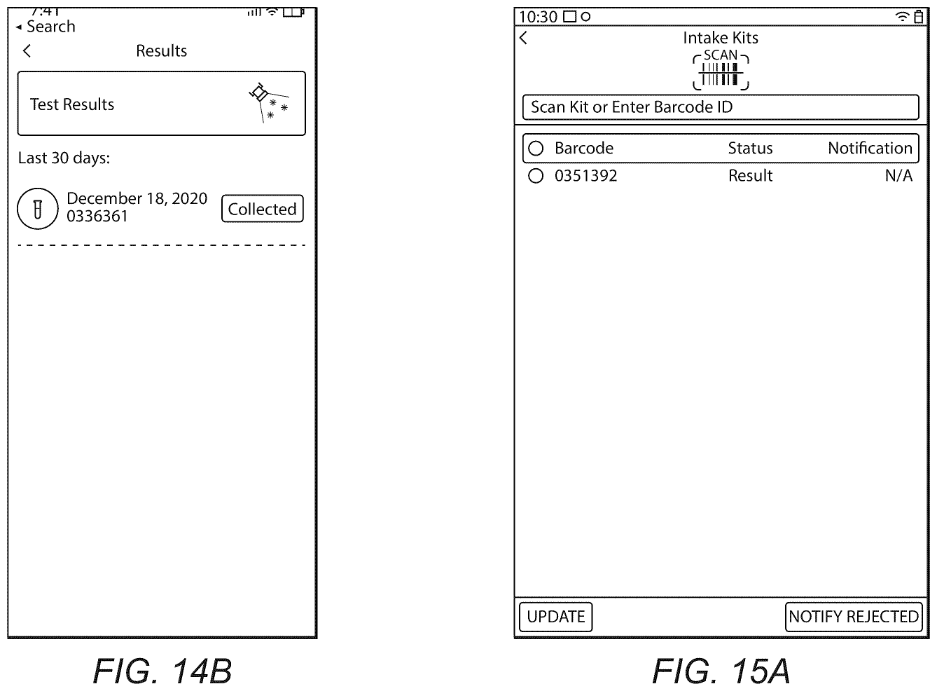

FIG. 14A

Example email notification

FIG. 14B

List displays historical collection events.

Intake (Staff)

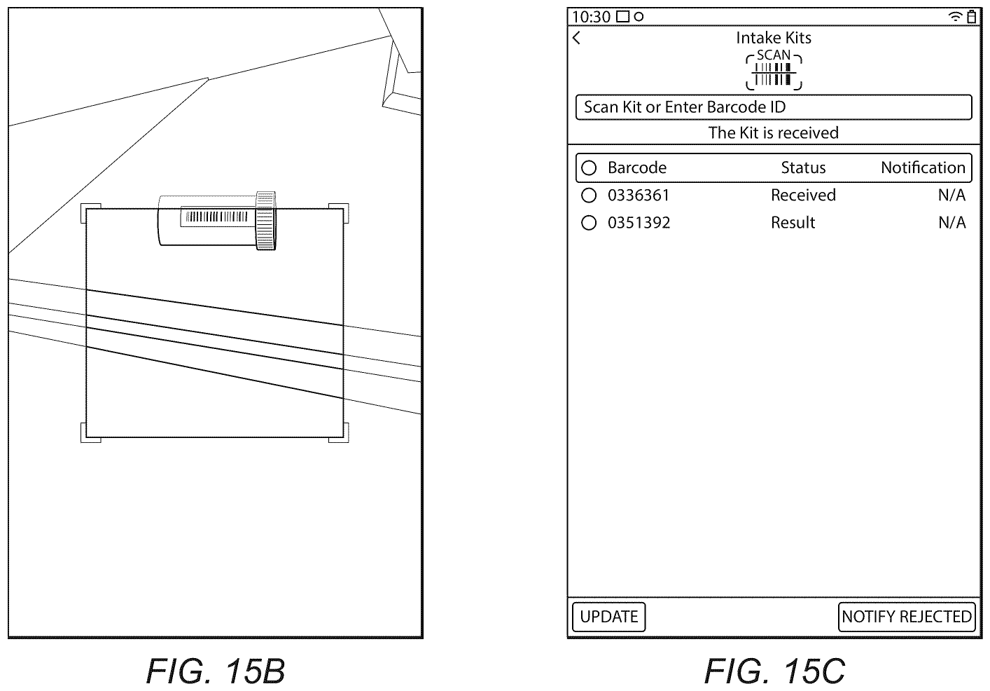

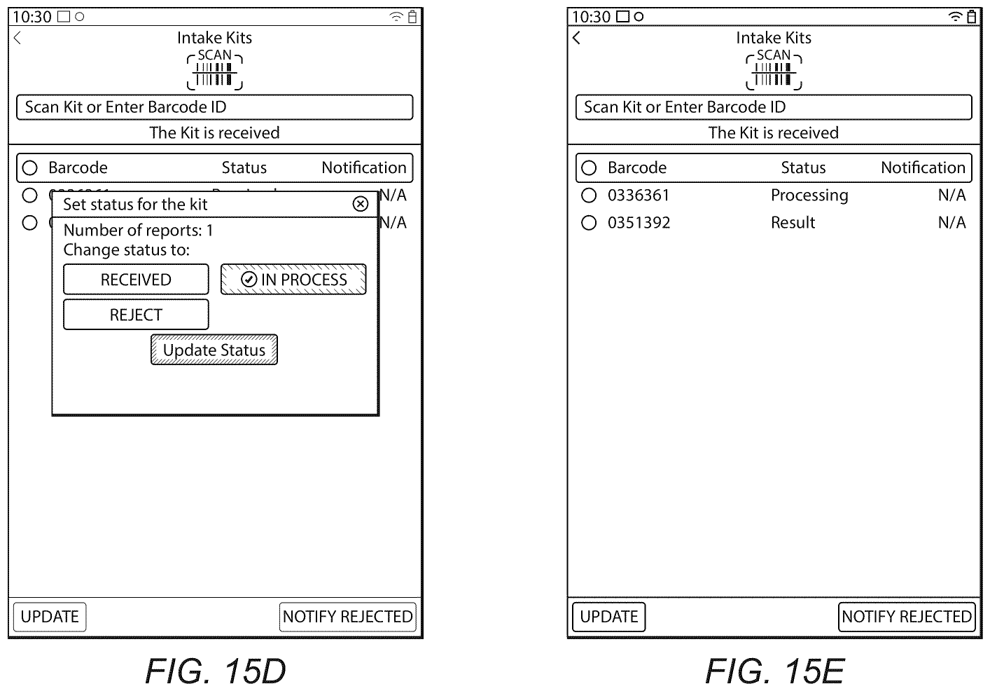

Staff members navigate to the INTAKE screen to add new kits to the system—see FIG. 15A. Options for adding are either through barcode scanning (see FIG. 15B) or manual entry of an identifier. Once added to the system, the staff member has the option to individually or batch select kits from the table and set their status—see FIGS. 15C-E.
Received—for the staff member to acknowledge the receipt of kits at collection points, for example, the kit status may be automatically set to “Received” once the barcode has been entered into the system. This indicates that the kit has been collected but is not yet being processed.
In Process—for the staff member, e.g., in the lab, may indicate that a given kit is in the lab and ready to run (in a test) or if the test is in progress.
Reject—the staff members may mark the status as “Reject” if samples in the kit are damaged or compromised.

Participants are able to view the status of their respective kits from within the Results screen of their app.

Results (Staff)

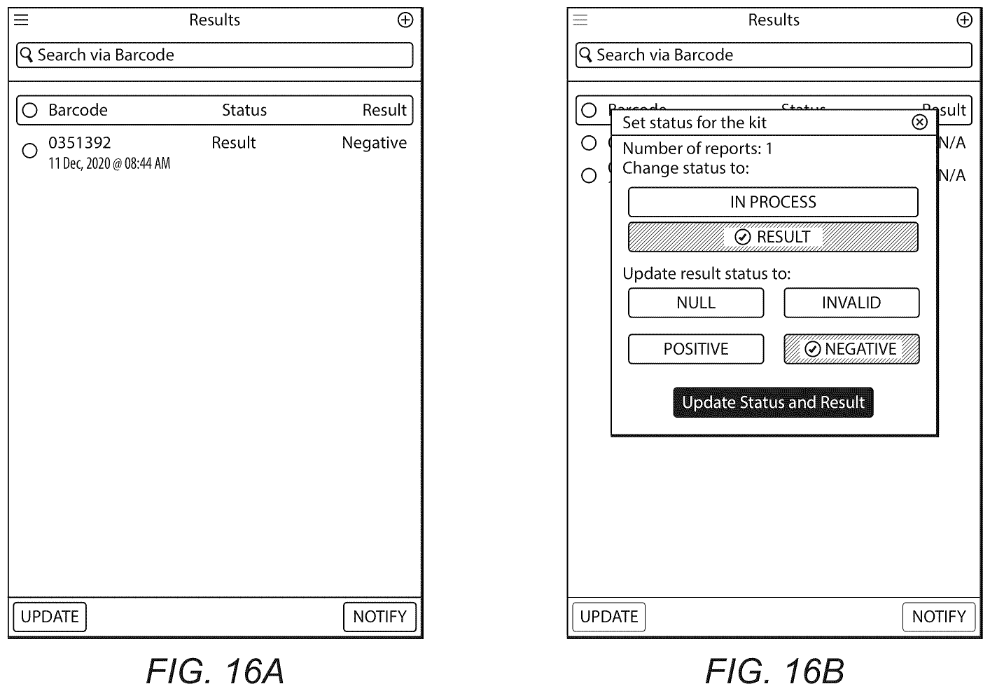

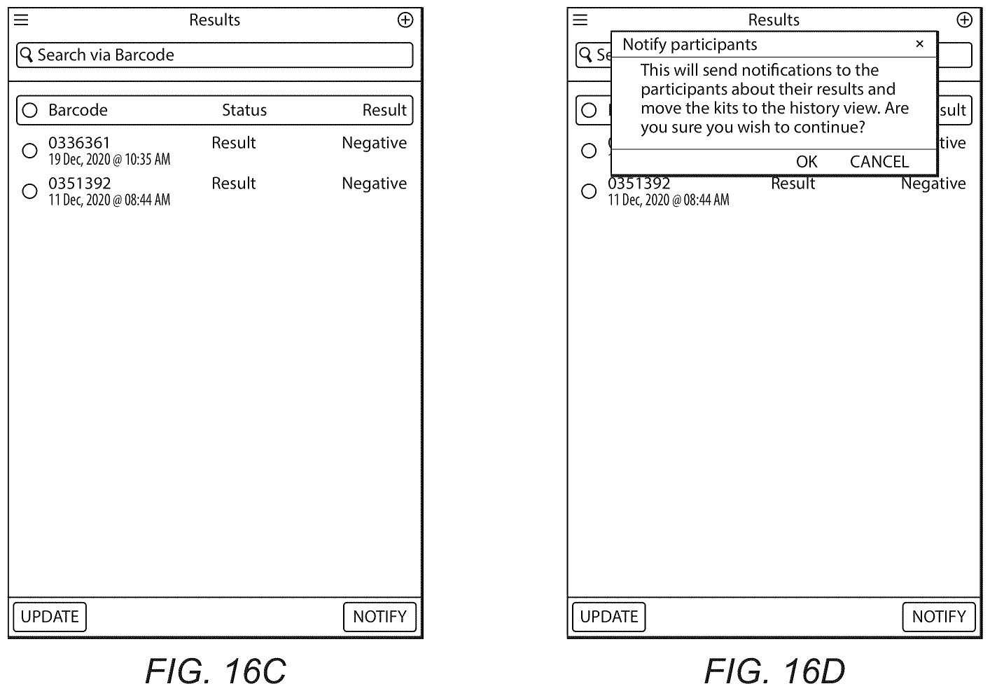

The Results view is the home view for the staff UI, and displays kits that are “In Process” or have been given a Result status—see FIG. 16A. There are four options for Results status (FIG. 16B):
1. Null—indicates that no Result has been yet set (this is the default setting for kits “In Process”)
2. Invalid—indicates that the Results are inconclusive or not readable
3. Positive—indicates a positive test result
4. Negative—indicates a negative test result

Kits remain in this view until they have the “Notify” action taken against them—see FIG. 16C. The “Notify” action will prompt a confirmation dialog that the staff member must OK prior to sending the notification to the respective participant(s)—see FIG. 16D. All messaging is handled on the backend. Once Notified, the kits are removed from this view and are available in the “History” view.

History (Staff)

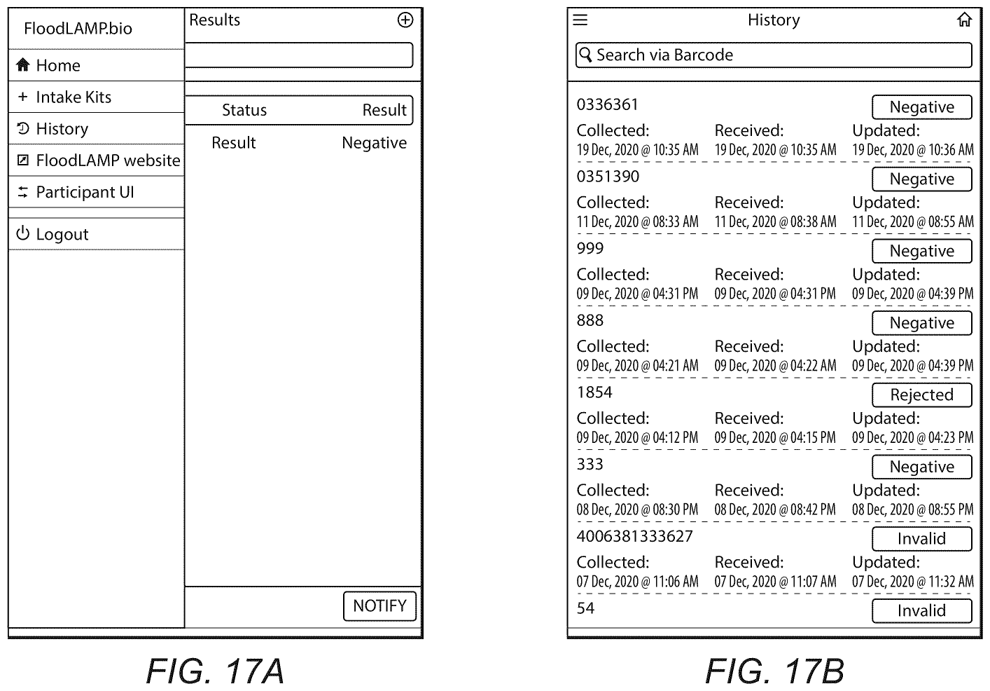

Historical test results are archived in the “History” view, which is accessed through the main menu—see FIG. 17A. Kits only appear in this view after a staff member has taken a “Notify” action against them—see FIG. 17B.

### Embodiment Set A—System

1. A system for registering a plurality of participants for a disease, diagnostic, or pathogen testing program, the system comprising:
one or more memories storing instructions;
one or more processors, operably coupled to the one or more memories, for executing the instructions to cause:
receiving, based on an input from a user, first participant information identifying a first participant the plurality of participants;
generating first registration information associated with the first participant based at least in part upon the first participant information;
receiving, from the user, second participant information identifying a second participant the plurality of participants;
generating second registration information associated with the second participant based at least in part upon the second participant information; and
providing at least a portion of the first registration information in response to a test request associated with a test to be performed, wherein the first participant is designated to participate in the test.

2. The system of embodiment 1, wherein the user and the first participant are the same individual.

3. The system of embodiment 1 or 2, wherein the first participant information or second participant information comprises relationship information indicating a relationship or an association between the first participant and the second participant.

4. The system of embodiment 3, wherein the relationship between the first participant and the second participant describes a family relationship including a spouse, a guardian, a child, a sibling, or a parent, and the association between the first participant and the second participant describes a housemate, a roommate, a care giver, a house guest, a co-worker, a proximity association based at least in part upon being within 10 m, 5 m, or 3 m of each other, or a co-occupancy association based at least in part upon occupying the same interior space of a structure.

5. The system of any one of the preceding embodiments, wherein the first participant provides a sample for the test at a sample collection event, and the instructions, when executed, cause: receiving a first set of information identifying one or more sample containers collected at the sample collection event, wherein at least one of the one or more sample containers includes the sample provided by the first participant; and
associating the portion of the first registration information and the first set of information.

6. The system of any one of the preceding embodiments, wherein at least a portion of the second registration information is provided in response to the test request, and the second participant is designated to participate in the test.

7. The system of embodiment 5 or 6, wherein the second participant provides a second sample for the test, the at least one of the one or more sample containers includes the second sample provided by the second participant, and the instructions, when executed, cause: associating at least a portion of the second registration information and the first set of information.

8. The system of any one of the preceding embodiments, wherein the instructions, when executed, cause:
providing the portion of the first registration information in response to a second test request associated with a second test to be performed, wherein the first participant is designated to participate in the second test, and the test and the second test are scheduled based on a testing schedule.

9. The system of any one of the preceding embodiments, wherein the instructions, when executed, cause:
providing test result information that causes display of a test result based on the test, wherein the test result is displayed on a computer system associated with the first participant.

10. The system of embodiment 9, wherein the test is performed on a pooled sample, the pooled sample includes samples from two or more participants including the first participant, and the test result information includes information identifying the two or more participants that provided samples for the test.

11. The system of any one of the preceding embodiments, wherein the first participant information or the second participant information comprises entity information associated with an entity.

12. The system of embodiment 11, wherein the entity information comprises an indication of an association between the entity and one or both of the first participant or the second participant.

13. The system of embodiment 11 or 12, wherein the entity information comprises an access code, a token, a QR code, or the like associated with the entity.

14. The system of any one of embodiments 11-13, where in the access code, the token, the QR code, or the like are provided by the entity.

15. The system of embodiment 13 or 14, wherein the access code, the token, the QR code, or the like comprises an indication of an association between the entity and one or both of the first participant or the second participant.

16. The system of any one of embodiments 12-15, wherein the first participant has an association with the entity, and the second participant does not have any association with the entity beyond any relationship or association they have to the first participant.

17. The system of any one of embodiments 12-16, wherein the association between the entity and the first participant or the second participant describes an employee, a student, a teacher, or a community member.

18. The system of any one of embodiments 11-17, wherein the testing program comprises two or more relationship/association groups, the first participant and the second participant are in a first relationship/association group of the two or more relationship/association groups, a third participant is in a second relationship/association group of the two or more relationship/association groups, the third individual has an association with the entity, individuals in the first relationship/association group provide samples for the test, individuals in the second relationship/association group provide samples for a third test, the entity receives a test result based on the test, the entity receives a third test result based on the third test, individuals in the first relationship/association group receive the test result and do not receive the third test result, and individuals in the second relationship/association group receive the third test result and do not receive the test result.

19. The system of any one of the preceding embodiments, wherein the first participant information and the second participant information are received based on entries made in a form accessed using a computing system.

20. The system of embodiment 19, wherein the form is a registration form available through a mobile application, a desktop application, a browser application, or via a website.

21. The system of any one of the preceding embodiments, wherein the first participant information and the second participant information are received in response to a single user action.

22. The system of any one of embodiments 1-20, wherein the first participant information and the second participant information are received less than 5 minutes, 1 hour, 8 hours, or 24 hours apart.

23. The system of any one of the preceding embodiments, wherein the second participant information is received separately from the first participant information, and the second participant information comprises relationship information indicating a relationship or an association between the first participant and the second participant.

24. The system of any one of embodiments 9-23, wherein the instructions, when executed, cause: providing follow-up information based at least in part upon the test result, wherein the follow-up information is displayed on the computer system.

25. The system of embodiment 24, wherein the follow-up information instructs the first participant to quarantine or provide another sample for testing.

26. The system of any one of embodiments 11-25, wherein the instructions, when executed, cause:
transmitting a registration request directed to the first participant or the second participant, wherein the registration request is based at least in part upon a request from the entity.

### Embodiment Set B—Method

1. A computer-implemented method for registering a plurality of participants for a disease, diagnostic, or pathogen testing program, the method comprising:
receiving, by a processor based on an input from a user, first participant information identifying a first participant the plurality of participants;
generating, by a processor, first registration information associated with the first participant based at least in part upon the first participant information;
receiving, by a processor from the user, second participant information identifying a second participant the plurality of participants;
generating, by a processor, second registration information associated with the second participant based at least in part upon the second participant information; and
providing, by a processor, at least a portion of the first registration information in response to a test request associated with a test to be performed, wherein the first participant is designated to participate in the test.

Note: Additional method embodiments include, at least and without limitation, the method described above in Embodiment Set B together with any combination of the system embodiments 2-26 from Embodiment Set A converted to a corresponding method form.

### Embodiment Set C—CRM

1. One or more non-transitory computer-readable media (CRM) storing instructions for registering a plurality of participants for a disease, diagnostic, or pathogen testing program, wherein the instructions, when executed by one or more computing devices, cause:
receiving, based on an input from a user, first participant information identifying a first participant the plurality of participants;
generating first registration information associated with the first participant based at least in part upon the first participant information;
receiving, from the user, second participant information identifying a second participant the plurality of participants;
generating second registration information associated with the second participant based at least in part upon the second participant information; and
providing at least a portion of the first registration information in response to a test request associated with a test to be performed, wherein the first participant is designated to participate in the test.

Note: Additional CRM embodiments include, at least and without limitation, the CRM described above in Embodiment Set C together with any combination of the system embodiments 2-26 from Embodiment Set A converted to a corresponding CRM form.

### Embodiment Set D—System

1. A system for providing a report for a disease, diagnostic, or pathogen testing program, the system comprising:
one or more memories storing instructions;
one or more processors, operably coupled to the one or more memories, for executing the instructions to cause:
identifying two or more participants participating in the testing program, wherein the report summarizes testing information related to the two or more participants, and the two or more participants includes a first participant and a second participant; and
providing first information that causes a display to display at least a portion of the report, wherein the portion of the report comprises an indication of a relationship or an association between the first participant and the second participant.

2. The system embodiment 1, wherein the relationship between the first participant and the second participant describes a family relationship including a spouse, a guardian, a child, a sibling, or a parent, and the association between the first participant and the second participant describes a housemate, a roommate, a care giver, a house guest, a co-worker, an association based at least in part upon being within 10 m, 5 m, or 3 m of each other, or an association based at least in part upon occupying the same interior space of a structure.

3. The system of embodiment 1 or 2, wherein the relationship or the association between the first participant and the second participant describes individuals who are in the same family, same household, or same exposure group.

4. The system of any one of the preceding embodiments, wherein the first participant and the second participant each provide a first sample to create a pooled sample for testing, and the instructions, when executed, cause:
receiving a first test result associated with a first test performed on the pooled sample, wherein the portion of the report comprises the first test result.

5. The system of any one of the preceding embodiments, wherein at least three or more participants are identified participating in the program, the three or more participants includes a third participant, the first participant and the third participant have an association with an entity, and the second participant does not have any association with the entity beyond any relationship or association they have to the first participant or the third participant.

6. The system of embodiment 5, wherein the association between the entity and the first participant or the third participant describes an employee, a student, a teacher, or a community member.

7. The system of embodiment 5 or 6, wherein the first participant is identified based at least in part upon their association with the entity.

8. The system of embodiment 7, wherein the second participant is identified based at least in part upon their relationship or association with the first participant.

9. The system of any one of embodiments 5-8, wherein the instructions, when executed, cause: receiving a second test result associated with a second test performed on a second sample, wherein the first participant contributes to the second sample; and
receiving a third test result associated with a third test performed on a third sample, wherein the third participant contributes to the third sample, the portion of the report comprises the second test result and the third test result, and the first information is provided to a computer system associated with the entity.

10. The system of embodiment 9, wherein the instructions, when executed, cause: providing second information that causes a second display to display at least a second portion of a second report, wherein the second portion of the second report comprises the indication of the relationship or the association between the first participant and the second participant, the second portion of the second report comprises the second test result, and the second information is provided to a computer system associated with the first participant.

11. The system of any one of the preceding embodiments, wherein the portion of the report comprises test results from two or more tests in which at least the first participant has participated.

12. The system of any one of the preceding embodiments, wherein the description of the relationship or the association between the first participant and the second participant is provided at the time of registration of the first participant or the second participant.

13. The system of any one of the preceding embodiments, wherein the first information is provided to a mobile application, a desktop application, a browser application, or via a website.

14. The system of any one of embodiments 4-13, wherein the instructions, when executed, cause: providing third information that causes the display to display follow-up information based at least in part upon the first test result or second test result, wherein the third information is provided to a computer system associated with the first participant.

15. The system of embodiment 14, wherein the follow-up information instructs the first participant to quarantine or provide another sample for testing.

### Embodiment Set E—Method

1. A computer-implemented method for providing a report for a disease, diagnostic, or pathogen testing program, the method comprising:
identifying two or more participants participating in the testing program, wherein the report summarizes testing information related to the two or more participants, and the two or more participants includes a first participant and a second participant; and
providing first information that causes a display to display at least a portion of the report, wherein the portion of the report comprises an indication of a relationship or an association between the first participant and the second participant.

Note: Additional method embodiments include, at least and without limitation, the method described above in Embodiment Set E together with any combination of the system embodiments 2-15 from Embodiment Set D converted to a corresponding method form.

### Embodiment Set F—CRM

1. One or more non-transitory computer-readable media (CRM) storing instructions for providing a report for a disease, diagnostic, or pathogen testing program, wherein the instructions, when executed by one or more computing devices, cause:
identifying two or more participants participating in the testing program, wherein the report summarizes testing information related to the two or more participants, and the two or more participants includes a first participant and a second participant; and
providing first information that causes a display to display at least a portion of the report, wherein the portion of the report comprises an indication of a relationship or an association between the first participant and the second participant.

Note: Additional CRM embodiments include, at least and without limitation, the CRM described above in Embodiment Set F together with any combination of the system embodiments 2-15 from Embodiment Set D converted to a corresponding CRM form.

### Embodiment Set G

1. A system for collecting information related to a sample collection event for pooled testing of a plurality of participants, the system comprising:
one or more memories storing instructions; and
one or more processors, operatively coupled to the one or more memories, for executing the instructions to cause the system to:
receive a first set of information identifying each of the plurality of participants, wherein the plurality of participants comprises a first participant and a second participant, and the first participant and the second participant are two different individuals;
receive a second set of information identifying one or more sample containers collected at the sample collection event, wherein a first specimen collected from the first participant and a second specimen collected from the second participant is contained in a first sample container of the one or more sample containers; and
associate the first set of information and the second set of information.

2. The system of embodiment 1, wherein the first specimen and the second specimen are in air or liquid contact with each other in the first sample container.

3. The system of any one of the preceding embodiments, wherein a first surface of the first sample container is in contact with the first specimen, and a second surface of the first sample container is in contact with the second specimen.

4. The system of any one of the preceding embodiments, wherein the first specimen collected from the first participant at a first time, the second specimen collected from the second participant at a second time, the first set of information is received at a third time, and the first, second, and third times span less than a first threshold period of time.

5. The system of embodiment 4, wherein the second set of information is received at a fourth time, and the first, second, third, and fourth times span less than the first threshold period of time.

6. The system of embodiment 4 or 5, wherein the first threshold period of time is less than: 10 minutes, 30 minutes, 1 hour, 2 hours, 8 hours, or 1 day.

7. The system of any one of the preceding embodiments, wherein the first specimen acquired from the first participant at a first location, the second specimen acquired from the second participant at a second location, the first specimen is added to the first container at a third location, the second specimen is added to the first container at a fourth location, and the first, second, third, and fourth location are within a threshold distance of each other.

8. The system of embodiment 7, wherein the threshold distance is less than: 10 feet, 30 feet, 50 feet, 100 feet, 300 feet, or 500 feet.

9. The system of any one of the preceding embodiments, wherein the instructions, when executed, cause the system to:
receive a first test result based at least in part upon a test performed on a first sample comprising at least a portion of the first specimen and at least a portion of the second specimen.

10. The system of embodiment 9, wherein the instructions, when executed, cause the system to:
transmit a notification to the first participant, wherein the notification is based at least in part upon the first test result.

11. The system of embodiment 10, wherein the notification comprises an indication that the first participant is requested to provide an additional specimen for additional testing.

12. The system of embodiment 11, wherein the additional testing is pooled sample testing.

13. The system of embodiment 11, wherein the additional testing is individual sample testing.

14. The system of any one of the preceding embodiments, wherein the instructions, when executed, cause the system to:
receive registration information related to the first participant, wherein the registration information comprises first identifying information, first participant identifying information is based at least in part upon the first identifying information, and the first set of information comprises the first participant identifying information.

15. The system of embodiment 14, wherein the registration information is provided by the first participant.

16. The system of embodiment 14 or 15, wherein the first identifying information is a name of the first participant, and the first participant identifying information is a unique identifier based at least in part upon the name.

17. The system of any one of embodiments 14, 15, and 16, wherein the registration information is received less than a second threshold period of time before the first set of information is received.

18. The system of embodiment 17, wherein the second threshold period of time is less than: 10 minutes, 30 minutes, 1 hour, 2 hours, 8 hours, or 1 day.

19. The system of any one of the preceding embodiments, wherein the first set of information and the second set of information are received together.

20. The system of any one of the preceding embodiments, wherein the association of the first set of information and the second set of information comprises generation of sample collection event information, the sample collection event information comprises a reference to at least a portion of the first set of information, and the sample collection event information comprises a reference to at least a portion of the second set of information.

21. The system of embodiment 20, wherein the sample collection event information comprises a reference to a time associated with the sample collection event.

22. The system of embodiment 20 or 21, wherein the sample collection event information comprises a reference to a location associated with the sample collection event.

23. The system of any one of the preceding embodiments, wherein the association of the first set of information and the second set of information comprises relating a first reference for at least a portion of the first set of information to a second reference for at least a portion of the second set of information.

24. The system of embodiment 23, wherein the first reference and the second reference are related in a data store.

25. The system of embodiment 24, wherein the first reference and the second reference are related using one or more tables in one or more databases.

26. The system of any one of the preceding embodiments, wherein the sample collection event spans a period of time including the collection of at least one specimen from each of the plurality of participants, and the period of time is less than a third threshold period of time.

27. The system of embodiment 26, wherein the third threshold period of time is less than: 10 minutes, 30 minutes, 1 hour, 2 hours, 8 hours, or 1 day.

28. The system of any one of the preceding embodiments, wherein the first set of information is based at least in part upon information received from a first user interface of a first application running on a first device associated with a first user.

29. The system of embodiment 28, wherein the first device is a smartphone, a tablet, a laptop, or a desktop.

30. The system of embodiment 28 or 29, wherein the first user has performed one or more actions required to manage at least a portion of the sample collection event.

31. The system of embodiment 30, wherein the first user has consented to follow at least one sample collection guideline.

32. The system of embodiment 30 or 31, wherein the first user has completed written or audio/video training.

33. The system of any one of embodiments 28-32, wherein the first participant and first user are the same person.

34. The system of any one of the preceding embodiments, wherein the second set of information is based at least in part upon information received from a second user interface of a second application running on a second device associated with a second user.

35. The system of embodiment 34, wherein the first participant and second user are the same person.

36. The system of any one of the preceding embodiments, wherein a second sample container of the one or more sample containers contains a third specimen collected from the first participant.

37. The system of embodiment 36, wherein a second test result is based at least in part upon a test performed on a second sample comprising at least a portion of the first specimen and at least a portion of the second specimen, and a third sample comprising the third specimen is tested based at least in part upon the second test result.

38. The system of any one of the preceding embodiments, wherein the first set of information is based at least in part upon first information related to a third participant from the plurality of participants, and the first information is collected during the sample collection event.

39. The system of any one of the preceding embodiments, wherein at least a portion of the sample collection event is monitored by a remote user.

40. The system of any one of the preceding embodiments, wherein at least a portion of the second set of information is based on information received by scanning a barcode associated with the first sample container during the sample collection event.

41. A method for collecting information related to a sample collection event for pooled testing of a plurality of participants, the method comprising:
receiving, by a processor, a first set of information identifying each of the plurality of participants, wherein the plurality of participants comprises a first participant and a second participant;
receiving, by a processor, a second set of information identifying one or more sample containers collected at the sample collection event, wherein a first specimen collected from the first participant and a second specimen collected from the second participant is contained in a first sample container of the one or more sample containers; and
associating, by a processor, the second set of information with the first set of information.

42. The method of embodiment 41, further comprising:
acquiring the first specimen from the first participant;
placing the first specimen into the first container;
acquiring the second specimen from the second participant; and
placing the second specimen into the first container.

43. The method of embodiment 41 or 42, further comprising:
receiving the first container at a collection facility.

44. The method of any one of embodiments 41, 42, and 43, further comprising:
testing a first sample comprising at least a portion of the first specimen and at least a portion of the second specimen.

45. The method of embodiment 44, further comprising:
receiving, by a processor, a first test result based at least in part upon a test performed on the first sample.

46. The method of any one of embodiments 41, 42, and 43, further comprising:
receiving, by a processor, a first test result based at least in part upon a test performed on a first sample comprising at least a portion of the first specimen and at least a portion of the second specimen.

47. The method of embodiment 45 or 46, further comprising:
transmitting, by a processor, a notification to the first participant, wherein the notification is based at least in part upon the first test result.

48. One or more non-transitory computer-readable media for collecting information related to a sample collection event for pooled testing of a plurality of participants, the one or more non-transitory computer-readable media storing instructions that, when executed by one or more computer systems, cause at least one of the one or more computer systems to:
receive a first set of information identifying each of the plurality of participants, wherein the plurality of participants comprises a first participant and a second participant; receive a second set of information identifying one or more sample containers collected at the sample collection event, wherein a first specimen collected from the first participant and a second specimen collected from the second participant is contained in a first sample container of the one or more sample containers; and
associate the second first set of information with and the first second set of information.

49. The system of embodiment 20, wherein the sample collection event information comprises a manifest identifier, and the manifest identifier is based at least in part upon the first set of information.

50. The system of embodiment 20 or 49, wherein the sample collection event information comprises a first container identifier, and the first container identifier is based at least in part upon the second set of information.

51. The system of embodiment 34, wherein the second participant and second user are the same person.

52. The system of any one of embodiments 1-40, wherein a third user maintains the identity of a fourth participant from the plurality of participants to permit anonymity of the fourth participant, and the identity of the fourth participant is tracked based at least in part upon de-identified information.

53. The system of any one of embodiments 1-40, wherein a fourth participant provides a specimen without registering with the system.

54. A system for collecting information related to a sample collection event for pooled testing of a plurality of participants, the system comprising:
one or more memories storing instructions; and
one or more processors, operatively coupled to the one or more memories, for executing the instructions to cause the system to:
receive first information identifying a first participant, wherein the plurality of participants comprises the first participant and a second participant, and the first participant and the second participant are two different individuals;
receive second information identifying a second participant;
receive third information identifying a first sample container, wherein a first specimen collected from the first participant and a second specimen collected from the second participant is contained in the first sample container; and
associate the first information, second information, and third information.

55. The system of embodiment 54, wherein the association of the first information, second information, and third information comprises generation of sample collection event information, the sample collection event information comprises a reference to at least a portion of the first information, the sample collection event information comprises a reference to at least a portion of the second information, and the sample collection event information comprises a reference to at least a portion of the third information.

56. The system of embodiment 55, wherein the sample collection event information comprises a manifest identifier.

57. A system for collecting information related to a sample collection event for pooled testing of a plurality of participants, the system comprising:
one or more memories storing instructions; and
one or more processors, operatively coupled to the one or more memories, for executing the instructions to cause the system to:
receive first information identifying a first participant, wherein the plurality of participants comprises the first participant and a second participant, and the first participant and the second participant are two different individuals;
receive second information identifying a second participant;
receive third information identifying a first sample container, wherein a first specimen collected from the first participant is contained in the first sample container;
receive fourth information identifying a second sample container, wherein a second specimen collected from the second participant is contained in the second sample container, and a portion of the first specimen and a portion of the second specimen are combined to create a pooled sample; and
associate the first information, second information, third information, and fourth information.

58. The system of embodiment 57, wherein the association of the first information, second information, third information, and fourth information comprises generation of pooled sample information, and the pooled sample information comprises a manifest identifier.

59. The system of embodiment 57 or 58, wherein the first information is received at a first time, the second information is received at a second time, the third information is received at a third time, the fourth information is received at a fourth time, the first information, second information, third information, and fourth information is associated at a fifth time, and the first time, second time, third time, fourth time, and fifth time span less than a threshold period of time.

60. The system of embodiment 59, wherein the threshold period of time is less than: 10 minutes, 30 minutes, 1 hour, 2 hours, 8 hours, or 1 day.

61. The system of embodiment 57, wherein the wherein the association of the first information, second information, third information, and fourth information comprises generation of pooled sample information, the pooled sample information comprises a manifest identifier, and an identifier without Personal Identifying Information is associated with the pooled sample.

62. The system of embodiment 61, wherein the identifier without Personal Identifying Information is used for tracking purposes during the pooled sample testing process.

63. The system of embodiment 58, wherein the first container and second container are bundled together in a third container, wherein the third container contains only specimens from each participant in a manifest referenced by the manifest identifier.

64. The system of embodiment 57, wherein the pooled sample is created after the sample collection event.

65. The system of embodiment 57, wherein a biomolecular or chemical test is performed on the pooled sample.

What is claimed is:

1. A system for registering a plurality of participants for a disease, diagnostic, or pathogen testing program, the system comprising:
one or more memories storing instructions;
one or more processors, operably coupled to the one or more memories, for executing the instructions to cause:
receiving, based on an input from a user, first participant information identifying a first participant the plurality of participants;
generating first registration information associated with the first participant based at least in part upon the first participant information;
receiving, from the user, second participant information identifying a second participant the plurality of participants;
generating second registration information associated with the second participant based at least in part upon the second participant information; and
providing at least a portion of the first registration information in response to a test request associated with a test to be performed, wherein the first participant is designated to participate in the test.

2. The system of claim 1, wherein the user and the first participant are the same individual.

3. The system of claim 1, wherein the first participant information or second participant information comprises relationship information indicating a relationship or an association between the first participant and the second participant.

4. The system of claim 3, wherein the relationship between the first participant and the second participant describes a family relationship including a spouse, a guardian, a child, a sibling, or a parent, and the association between the first participant and the second participant describes a housemate, a roommate, a care giver, a house guest, a co-worker, a proximity association based at least in part upon being within 10 m, 5 m, or 3 m of each other, or a co-occupancy association based at least in part upon occupying the same interior space of a structure.

5. The system of claim 1, wherein the first participant provides a sample for the test at a sample collection event, and the instructions, when executed, cause:
receiving a first set of information identifying one or more sample containers collected at the sample collection event, wherein at least one of the one or more sample containers includes the sample provided by the first participant; and
associating the portion of the first registration information and the first set of information.

6. The system of claim 5, wherein the second participant provides a second sample for the test, the at least one of the one or more sample containers includes the second sample provided by the second participant, and the instructions, when executed, cause:
associating at least a portion of the second registration information and the first set of information.

7. The system of claim 1, wherein the instructions, when executed, cause:
providing the portion of the first registration information in response to a second test request associated with a second test to be performed, wherein the first participant is designated to participate in the second test, and the test and the second test are scheduled based on a testing schedule.

8. The system of claim 1, wherein the instructions, when executed, cause:
providing test result information that causes display of a test result based on the test, wherein the test result is displayed on a computer system associated with the first participant, the test is performed on a pooled sample, the pooled sample includes samples from two or more participants including the first participant, and the test result information includes information identifying the two or more participants that provided samples for the test.

9. The system of claim 1, wherein the first participant information or the second participant information comprises entity information associated with an entity, and the entity information comprises an indication of an association between the entity and one or both of the first participant or the second participant.

10. The system of claim 9, wherein the first participant has an association with the entity, and the second participant does not have any association with the entity beyond any relationship or association they have to the first participant.

11. The system of claim 9, wherein the testing program comprises two or more relationship/association groups, the first participant and the second participant are in a first relationship/association group of the two or more relationship/association groups, a third participant is in a second relationship/association group of the two or more relationship/association groups, the third individual has an association with the entity, individuals in the first relationship/association group provide samples for the test, individuals in the second relationship/association group provide samples for a third test, the entity receives a test result based on the test, the entity receives a third test result based on the third test, individuals in the first relationship/association group receive the test result and do not receive the third test result, and individuals in the second relationship/association group receive the third test result and do not receive the test result.

12. The system of claim 9, wherein the instructions, when executed, cause:
transmitting a registration request directed to the first participant or the second participant, wherein the registration request is based at least in part upon a request from the entity.

13. The system of claim 1, wherein the first participant information and the second participant information are received based on entries made in a form accessed using a computing system.

14. The system of claim 1, wherein the first participant information and the second participant information are received in response to a single user action.

15. The system of claim 1, wherein the second participant information is received separately from the first participant information, and the second participant information comprises relationship information indicating a relationship or an association between the first participant and the second participant.

16. A system for providing a report for a disease, diagnostic, or pathogen testing program, the system comprising:
one or more memories storing instructions;
one or more processors, operably coupled to the one or more memories, for executing the instructions to cause:
identifying two or more participants participating in the testing program, wherein the report summarizes testing information related to the two or more participants, and the two or more participants includes a first participant and a second participant; and
providing first information that causes a display to display at least a portion of the report, wherein the portion of the report comprises an indication of a relationship or an association between the first participant and the second participant.

17. The system of claim 16, wherein the first participant and the second participant each provide a first sample to create a pooled sample for testing, and the instructions, when executed, cause:
receiving a first test result associated with a first test performed on the pooled sample, wherein the portion of the report comprises the first test result.

18. The system of claim 16, wherein at least three or more participants are identified participating in the program, the three or more participants includes a third participant, the first participant and the third participant have an association with an entity, and the second participant does not have any association with the entity beyond any relationship or association they have to the first participant or the third participant.

19. The system of claim 16, wherein the portion of the report comprises test results from two or more tests in which at least the first participant has participated.

20. The system of claim 16, wherein the description of the relationship or the association between the first participant and the second participant is provided at the time of registration of the first participant or the second participant.
